(19) United States
(12) Patent Application Publication
(10) Pub. No.: US 2026/0265403 A1
(43) Pub. Date: Sep. 10, 2026

(54) ANTI-CD24 ANTIBODIES AND USES THEREOF

(71) Applicant: TJ BIOPHARMA (SHANGHAI) CO., LTD., Shanghai (CN)

(72) Inventors: Xue LI, Shanghai (CN); Xi CHEN, Shanghai (CN); Pan HU, Shanghai (CN); Zhengyi WANG, Shanghai (CN); Guiliang XU, Shanghai (CN)

(21) Appl. No.: 19/491,273

(22) PCT Filed: Jun. 7, 2024
(86) PCT No.: PCT/CN2024/098071

§ 371 Date: Dec. 8, 2025

(30) Foreign Application Priority Data
Jun. 9, 2023 (WO) PCT/CN2023/099343

(51) Int. Cl. C07K 16/28; A61P 35/00

(52) U.S. Cl. CPC C07K 16/2896; A61P 35/00; C07K 2317/24

(57) ABSTRACT

Provided are antibodies or fragments thereof having binding specificity to the human CD24 protein. In various examples, the antibodies or fragments thereof include a VH and VL CDRs as disclosed herein, or variants thereof. Methods of using the antibodies or fragments thereof for treating cancer are also provided.

### BACKGROUND

CD24 is a small, heavily glycosylated protein attached to the cell membrane by a glycosyl-phosphatidylinositol (GPI) anchor. It contains three potential N-glycosylation sites and several potential O-glycosylation sites resulting in that CD24 protein isolated from different tissues or cell types has different molecular weights, ranging from 20 to 70 kDa.

CD24 plays an important role in tumorigenesis. Surface CD24 expression in tumor cells has been linked with alterations in multiple oncogenic signaling pathways, including Src/STAT3, EGFR, HER2, Ras-like GTPase, MAPK, AKT/mTOR, WNT/β-catenin, and miRNA-related pathways. Moreover, the cytoplasmic accumulation of CD24 may be involved in tumor cell proliferation, including p53 inactivation. In this regard, the overexpression of CD24 has been documented in several malignancies, including breast, lung, colorectal, hepatocellular, pancreatic, ovarian, urothelial, prostate, and head and neck cancer, as well as in primary central nervous system (CNS) tumors and several hematologic malignancies. The expression of CD24 in solid tumors has been correlated with worse prognosis, including the presence of more aggressive features and their metastatic spread. In addition, CD24 is also described as one of the most important cancer stem cell markers.

Recent studies showed that the surface expression of CD24 can serve as a “do not eat me” signal, inhibiting phagocytosis by infiltrating Siglec-10-expressing macrophages. In contrast, the loss of CD24 expression has been shown to increase phagocytosis by macrophages expressing Siglec-10 in several tumor types.

There is a need for targeting CD24 in cancer therapy.

### SUMMARY

The present disclosure provides antibodies or fragments thereof having binding specificity to the human CD24 protein. These antibodies and fragments are useful in the treatment of cancers and infectious diseases.

Accordingly, one embodiment of the present disclosure provides an antibody or antigen-binding fragment thereof having specificity to a human CD24 protein, wherein the antibody or antigen-binding fragment thereof comprises a heavy chain variable region comprising heavy chain complementarity determining regions HCDR1, HCDR2, and HCDR3, and a light chain variable region comprising light chain complementarity determining regions LCDR1, LCDR2, and LCDR3, wherein the HCDR1, HCDR2, HCDR3, LCDR1, LCDR2, and LCDR3 comprise, respectively: HCDR1: GYHMN (SEQ ID NO: 1) or GYHMG (SEQ ID NO: 112), HCDR2: EINPITSDKTFNQKFKS (SEQ ID NO: 2) or EINPITSDKYFNQKFKS (SEQ ID NO:113), HCDR3: RDYGTSLDY (SEQ ID NO: 3), LCDR1: RASKSISKYLA (SEQ ID NO: 4), RASASISKYLA (SEQ ID NO: 114), RASKSIKKYLA (SEQ ID NO: 115) or RASKSISKYGA (SEQ ID NO: 116), LCDR2: AGSTLHS (SEQ ID NO: 5), and LCDR3: QQHNEYPIT (SEQ ID NO: 6) or QQHNEYPII (SEQ ID NO: 117).

In some embodiments, the antibody is a chimeric antibody or a humanized antibody.

In some embodiments, the HCDR1, HCDR2, HCDR3, LCDR1, LCDR2, and LCDR3 are HCDR1: GYHMG (SEQ ID NO: 112), HCDR2: EINPITSDKYFNQKFKS (SEQ ID NO: 113), HCDR3: RDYGTSLDY (SEQ ID NO: 3), LCDR1: RASASISKYLA (SEQ ID NO: 114), LCDR2: AGSTLHS (SEQ ID NO: 5), and LCDR3: QQHNEYPIT (SEQ ID NO: 6).

In some embodiments, the antibody or antigen-binding fragment thereof is humanized and wherein the heavy chain variable region comprises one or more back mutations selected from the group consisting of 38K, 48I, 68A, 72V, and 74R, according to Kabat numbering, and combinations thereof.

In some embodiments, the antibody or antigen-binding fragment thereof is humanized and wherein the light chain variable region comprises one or more back mutations selected from the group consisting of 45R, 58I, and 63R according to Kabat numbering, and combinations thereof.

In some embodiments, the antibody or antigen-binding fragment thereof comprises a heavy chain variable region comprising an amino acid sequence selected from the group consisting of SEQ ID NOs: 60, 94-97, 102, 110, and 111, or a peptide having at least 90% sequence identity to an amino acid sequence selected from the group consisting of SEQ ID NOs: 60, 94-97, 102, 110, and 111.

In some embodiments, the antibody or antigen-binding fragment thereof comprises a light chain variable region comprising an amino acid sequence selected from the group consisting of SEQ ID NOs: 61, 98-101, and 103-109 or a peptide having at least 90% sequence identity to an amino acid sequence selected from the group consisting of SEQ ID NOs: 61, 98-101, and 103-109.

In some embodiments, the antibody or antigen-binding fragment thereof comprises a heavy chain variable region comprising the amino acid sequence of SEQ ID NO: 111 or a peptide having at least 90% sequence identity to SEQ ID NO: 111, and a light chain variable region comprising the amino acid sequence of SEQ ID NO: 106 or a peptide having at least 90% sequence identity to SEQ ID NO: 106.

In some embodiments, the antibody or antigen-binding fragment thereof comprises a heavy chain variable region comprising the amino acid sequence of SEQ ID NO: 111, and a light chain variable region comprising the amino acid sequence of SEQ ID NO: 106.

Also provided, in one embodiments, is an antibody or antigen-binding fragment thereof having specificity to a human CD24 protein, wherein the antibody or antigen-binding fragment thereof comprises a heavy chain variable region comprising heavy chain complementarity determining regions HCDR1, HCDR2, and HCDR3, and a light chain variable region comprising light chain complementarity determining regions LCDR1, LCDR2, and LCDR3, wherein: the set of HCDR1, HCDR2, and HCDR3 is selected from Table 1, or CDR sets derived from Table 1 or Table 5 with one, two, or three amino acid addition, deletion and/or substitution in one or more of the CDRs, and the set of LCDR1, LCDR2, and LCDR3 are selected from Table 1 or CDR sets derived from Table 1 or Table 5 with one, two, or three amino acid addition, deletion and/or substitution in one or more of the CDRs.

Another embodiment provides an antibody or antigen-binding fragment thereof having specificity to a human CD24 protein, wherein the antibody or antigen-binding fragment thereof competes with the antibody or antigen-binding fragment thereof of any one of the preceding claims in binding to the CD24 protein.

In some embodiments, the antibody or antigen-binding fragment thereof further comprises a heavy chain constant region, a light chain constant region, an Fc region, or the combination thereof.

In some embodiments, the light chain constant region is a kappa or lambda chain constant region.

In some embodiments, the antibody or antigen-binding fragment thereof is of an isotype of IgG, IgM, IgA, IgE or IgD. In some embodiments, the isotype is IgG1, IgG2, IgG3 or IgG4. In some embodiments, the Fc region is engineered to enhance the effector function.

In some embodiments, the effector function comprises antibody-mediated cellular cytotoxicity (ADCC), complement dependent cytotoxicity (CDC) or antibody-dependent cellular phagocytosis (ADCP). In some embodiments, the Fc region is engineered to include S298A/E333A/K334A mutations in IgG1.

Further provided is a bifunctional molecule, comprising a first antigen-binding portion having specificity to a human CD24 protein and a second portion having specificity to a second protein, wherein the first antigen-binding portion comprises an antibody or antigen-binding fragment thereof of the present disclosure.

Still further provided is a composition comprising the antibody or antigen-binding fragment thereof or the bifunctional molecule of the present disclosure, and a pharmaceutically acceptable carrier.

In yet another embodiment, provided is an isolated cell comprising one or more polynucleotide encoding the antibody or antigen-binding fragment thereof or the bifunctional molecule of the present disclosure.

In yet another embodiment, provided polynucleotide encoding one or more chains of the antibody or antigen-binding fragment thereof or the bifunctional molecule.

Methods of treating a cancer in a patient in need thereof are also provided, comprising administering to the patient the antibody or antigen-binding fragment thereof or the bifunctional molecule of the present disclosure.

In some embodiments, the cancer is selected from the group consisting of bladder cancer, breast cancer, colorectal cancer, endometrial cancer, esophageal cancer, head and neck cancer, kidney cancer, leukemia, liver cancer, lung cancer, lymphoma, melanoma, pancreatic cancer, prostate cancer, and thyroid cancer.

In some embodiments, the method further comprises administering to the patient a second therapy for treating said cancer.

In some embodiments, said therapy is selected from the group consisting of immunotherapy, chemotherapy and radiotherapy.

Also provided is a method for treating an autoimmune disease or inflammatory condition in a patient in need thereof, comprising administering to the patient the antibody or antigen-binding fragment thereof or the bifunctional molecule of the present disclosure.

In some embodiments, the disease is characterized with CD24 expression.

Still further provided is a method of detecting expression of CD24 in a sample, comprising contacting the sample with the antibody or antigen-binding fragment thereof under conditions for the antibody or antigen-binding fragment thereof to bind to the CD24, and detecting the binding which indicates expression of CD24 in the sample.

### BRIEF DESCRIPTION OF THE DRAWINGS

FIG. 1 shows the results of phagocytosis (panel A) and tumor cell binding assays (panel B) performed using the selected clones fused with human IgG4: 89-E8C12 (4428), 74-H4C6 (4451) and 85-D5A10 (4434), with anti-CD24 antibodies (NS-17 (SWA11), pp 6373 and SN3) and IgG isotypes.

FIG. 2 shows the results of the tumor cell binding affinity (panel A and B) and phagocytosis (panel C) of the 16 humanized 4451.

FIG. 3 shows the results of the 4451H-IgG4 in the affinity binding by Biacore (panel A and B), tumor cells binding (panel C), CD24-Siglec-10 blocking (panel D) and phagocytosis (panel E) as compared with reference antibodies.

FIG. 4 shows tumor regression in Siglec-10 humanized mice treated with anti-CD24 antibodies. (Panel A) hCD24-MC38 cells were subcutaneously implanted into huSiglec-10 mice. When tumor grew to an average of 80 mm3, mice (n=6/group) were intraperitoneally treated with PBS, 4451H-IgG4, 4451H-IgG1-3A, GB7011-IgG4 and IMM47H at dose of 10 mg/kg twice weekly for 6 doses. Tumor growth was monitored by volumetric measurement. (Panel B) Tumor growth curve. Shown are mean±SEM of tumor volume. (Panel C) Body weight growth curve. (Panel D) Individual tumor growth curve. (Panel E) Tumor weight at sacrifice.

FIG. 5 shows the results of tumor cells binding affinity (panel A and B), phagocytosis (panel C), CD24 and siglec 10 block assay (panel D) of the 18 affinity matured antibodies.

FIG. 6 shows binding results of anti-CD24 mAbs to soluble CD24 as measure by ELISA. Concentration-dependent binding of 4451HM15-IgG1-3A, GB7011 and IMM47H to CD24-his by ELISA.

FIG. 7 shows the cell-based binding of anti-CD24 mAbs. Antibodies were 4-fold serially diluted from 40 μg/mL and incubated with MCF7 cells (panel A), OVCAR3 cells (panel B), A549 cells (panel C), and NALM6 cells (panel D) cells for 30 minutes at 4° C. in FACS buffer. Then, PE conjugated-anti-human IgG (H+L) antibody was added and incubated for another 30 minutes. After washing, geometric mean fluorescence intensity (GMFI) of PE was evaluated by FACS.

FIG. 8 shows the blocking effect of CD24/Siglec-10 interaction by anti-CD24 mAbs. Siglec-10-his-Biotin were mixed together with Anti-CD24 mAb of different concentrations and then added to the plate coated with CD24 protein, incubated for 1 hour. Samples were washed three times with washing buffer. And then signal was tested.

FIG. 9 shows anti-CD24 mAb-mediated phagocytosis of MCF7, OVCAR3 or NALM6 cells by macrophages. MCF7 (panel A), OVCAR3 (panel B), NALM6 (panel C) or MC38 cells (panel D) were labelled with CFSE and incubated with human peripheral blood-derived macrophages in the presence of different concentrations of anti-CD24 mAbs or isotype control. One hour later, macrophages were collected for flow cytometry analysis. Phagocytosis index was calculated by quantitating the percent of CFSE⁺ cells in CD14⁺ cells, normalized to that of IgG control.

FIG. 10 shows the ADCC and CDC activity of anti-CD24 mAbs against MCF7. MCF7 cells were incubated with increasing concentration of the indicated antibodies and ADCC (panel A) and CDC (panel B) activity was performed by incubation with PBMC or complement factor. LDH method was applied to detect the killing activity.

FIG. 11 shows tumor regression in Siglec-10 humanized mice treated with anti-CD24 antibodies. (Panel A) hCD24-MC38 cells were subcutaneously implanted into huSiglec-10 mice. When tumor grew to an average of 80 mm3, mice (n=6/group) were intraperitoneally treated with PBS, and 4451HM15-IgG1-3A, at dose of 10 mg/kg twice weekly for 6 doses. Tumor growth was monitored by volumetric measurement. (Panel B) Tumor growth curve. Shown are mean±SEM of tumor volume. (Panel C) Tumor weight at sacrifice. (Panel D) Tumor volume growth curve with PBS. (Panel E) Tumor volume growth curve with 4451HM15-IgG1-3A.

FIG. 12 shows the tumor regression in wt C57BL/6 mice treated with anti-CD24 antibodies. (Panel A) hCD24-MC38 cells were subcutaneously implanted into mice. When tumor grew to an average of 50 mm3, mice (n=6/group) were intraperitoneally treated with PBS or 4451HM15-IgG1-3A twice weekly for 6 doses. Tumor growth was monitored by volumetric measurement; (Panel B) Tumor growth curve. Shown are mean±SEM of tumor volume; (Panel C) Individual tumor growth curve treated with PBS; (Panel D) Individual tumor growth curve treated with 4451HM15-IgG1-3A.

FIG. 13 shows the tumor inhibition of anti-CD24 mAb in humanized PBMC xenograft model. (Panel A) MCF7 cells were subcutaneously inoculated into SCID mice. After 6 days, mice (n=6/group) were implanted with human PBMC, 5 million/mouse. And then, mice were intraperitoneally administrated with PBS or antibodies Q3D for three weeks. The tumor growth was monitored by volumetric measurement. (Panel B) Tumor growth curve. Shown are mean±SEM of tumor volume. (Panel C) Mice weight growth curve. (Panel D) Tumor weight at sacrifice.

FIG. 14 (panels A-B) shows the in vivo efficacy of the humanized anti-CD24 antibody 4451HM15-IgG1-3A in the SHP-77 tumor cell bearing CB-17 SCID mice.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

A “single-chain variable fragment” or “scFv” refers to a fusion protein of the variable regions of the heavy (V_{H}) and light chains (V_{L}) of immunoglobulins. In some aspects, the regions are connected with a short linker peptide of ten to about 25 amino acids. The linker can be rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker. ScFv molecules are known in the art and are described, e.g., in U.S. Pat. No. 5,892,019.

The term antibody encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgG₅, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgAQ1 and IgA2) or subclass of immunoglobulin molecule.

Light chains are classified as either kappa or lambda (K, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VK) and heavy (VH) chain portions determine antigen recognition and specificity. Conversely, the constant domains of the light chain (CK) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region; the CH3 and CK domains actually comprise the carboxy-terminus of the heavy and light chain, respectively.

As indicated above, the variable region allows the antibody to selectively recognize and specifically bind epitopes on antigens. That is, the VK domain and V_{H} domain, or subset of the complementarity determining regions (CDRs), of an antibody combine to form the variable region that defines a three dimensional antigen-binding site. This quaternary antibody structure forms the antigen-binding site present at the end of each arm of the Y. More specifically, the antigen-binding site is defined by three CDRs on each of the V_{H} and VK chains (i.e. CDR-H1, CDR-H2, CDR-H3, CDR-L1, CDR-L2 and CDR-L3). In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen-binding domain are short, non-contiguous sequences of amino acids that are specifically positioned to form the antigen-binding domain as the antibody assumes its three dimensional configuration in an aqueous environment. The remainder of the amino acids in the antigen-binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see “Sequences of Proteins of Immunological Interest,” Kabat, E., et al., U.S. Department of Health and Human Services, (1983); and Chothia and Lesk, J. Mol. Biol., 196:901-917 (1987)).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

Antibodies disclosed herein may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks).

As used herein, the term “heavy chain constant region” includes amino acid sequences derived from an immunoglobulin heavy chain. A polypeptide comprising a heavy chain constant region comprises at least one of: a CH1 domain, a hinge (e.g., upper, middle, and/or lower hinge region) domain, a CH2 domain, a CH3 domain, or a variant or fragment thereof. For example, an antigen-binding polypeptide for use in the disclosure may comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the disclosure comprises a polypeptide chain comprising a CH3 domain. Further, an antibody for use in the disclosure may lack at least a portion of a CH2 domain (e.g., all or part of a CH2 domain). As set forth above, it will be understood by one of ordinary skill in the art that the heavy chain constant region may be modified such that they vary in amino acid sequence from the naturally occurring immunoglobulin molecule.

The heavy chain constant region of an antibody disclosed herein may be derived from different immunoglobulin molecules. For example, a heavy chain constant region of a polypeptide may comprise a CH1 domain derived from an IgG₁ molecule and a hinge region derived from an IgG₃ molecule. In another example, a heavy chain constant region can comprise a hinge region derived, in part, from an IgG₁ molecule and, in part, from an IgG₃ molecule. In another example, a heavy chain portion can comprise a chimeric hinge derived, in part, from an IgG₁ molecule and, in part, from an IgG₄ molecule.

As used herein, the term “light chain constant region” includes amino acid sequences derived from antibody light chain. Preferably, the light chain constant region comprises at least one of a constant kappa domain or constant lambda domain.

A “light chain-heavy chain pair” refers to the collection of a light chain and heavy chain that can form a dimer through a disulfide bond between the CL domain of the light chain and the CH1 domain of the heavy chain.

As previously indicated, the subunit structures and three dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat et al., U.S. Dept. of Health and Human Services, “Sequences of Proteins of Immunological Interest” (1983). The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

As used herein, the term “hinge region” includes the portion of a heavy chain molecule that joins the CH1 domain to the CH2 domain. This hinge region comprises approximately 25 residues and is flexible, thus allowing the two N-terminal antigen-binding regions to move independently. Hinge regions can be subdivided into three distinct domains: upper, middle, and lower hinge domains (Roux et al., J. Immunol 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CK regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” will be held to mean any antibody wherein the immunoreactive region or site is obtained or derived from a first species and the constant region (which may be intact, partial or modified in accordance with the instant disclosure) is obtained from a second species. In certain embodiments the target binding region or site will be from a non-human source (e.g. mouse or primate) and the constant region is human.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

### Anti-CD24 Antibodies

The present disclosure provides antibodies, including antibodies or antigen-binding fragments thereof, that have binding specificity to the human CD24 protein. As demonstrated in the experimental examples, numerous murine anti-human CD24 antibodies were obtained, having high binding affinity to the human CD24 protein. The murine antibody clone, 74-H4C6, was selected for further humanization and characterization. The humanized antibodies bound human CD24 with high affinity and efficiently blocked the binding between CD24 and Siglec-10.

Additional functional studies showed that these antibodies or antigen-binding fragments thereof efficiently bound to a broad groups of cancer cells with potent activity and enhanced macrophage-mediated phagocytosis against multiple tumor cells. These antibodies significantly suppressed tumor growth in vivo.

In accordance with one embodiment of the present disclosure, provided are antibodies or antigen-binding fragments thereof that include the heavy chain and light chain variable domains with the CDR regions of the antibodies prepared in the experimental examples. The CDRs are summarized in Table 1A below.

TABLE 1A
CDR sequences of anti-CD24 antibody clones

| Antibody chain | Sequences | | SEQ ID NO: |
|---|---|---|---|
| 74-H4C6-VH | CDR1 | GYHMN | 1 |
| | CDR2 | EINPITSDKTFNQKEKS | 2 |
| | CDR3 | RDYGTSLDY | 3 |
| 74-H4C6-VL | CDR1 | RASKSISKYLA | 4 |
| | CDR2 | AGSTLHS | 5 |
| | CDR3 | QQHNEYPIT | 6 |
| 76-F8C6-VH | CDR1 | NSWMN | 15 |
| | CDR2 | QIQLKSDNYATRYVESV | 16 |
| | CDR3 | YYCTTGTDY | 17 |
| 76-F8C6-VL | CDR1 | KSSQSLLFSNGKTYLN | 18 |
| | CDR2 | LVSNLDS | 19 |
| | CDR3 | VQGTHEPYT | 20 |
| 59-G9D7-VH | CDR1 | TYWMH | 21 |
| | CDR2 | RIDPNSGETKYSEKFKN | 22 |
| | CDR3 | YYYLNNFLFDY | 23 |
| 59-G9D7-VL | CDR1 | RASSSVSSSYLH | 24 |
| | CDR2 | STSNLAS | 25 |
| | CDR3 | QQYSGYPLT | 26 |
| 75-H8B9-VH | CDR1 | KHWMN | 27 |
| | CDR2 | QIQLKSDNYAIRYVDSV | 28 |
| | CDR3 | YYCTTGTDY | 17 |
| 75-H8B9-VL | CDR1 | RSSQSLLYSNGKTYLN | 29 |
| | CDR2 | VVSNLDS | 30 |
| | CDR3 | VQGTHEPYT | 20 |
| 62-F7E9-VH | CDR1 | SYWLH | 31 |
| | CDR2 | RIDPNSGETKFNEKFKS | 32 |
| | CDR3 | YYYGSILFDY | 33 |
| 62-F7E9-VL | CDR1 | RTSSIISSGFLH | 34 |
| | CDR2 | STSNLAS | 25 |
| | CDR3 | QQYSGHPLT | 35 |
| 45-H1E4-VH | CDR1 | TYGMS | 36 |
| | CDR2 | WINTYSGVPAYADDFKG | 37 |
| | CDR3 | SGYPWSPHSYSTDY | 38 |
| 45-H1E4-VL | CDR1 | KSSQSIVHSNGNTYLE | 39 |
| | CDR2 | KVSNRFS | 40 |
| | CDR3 | FQGSHVPWT | 41 |
| 79-E3H3-VH | CDR1 | GYHMN | 12 |
| | CDR2 | EINPITSDKTFNQKFKA | 13 |
| | CDR3 | RDYDTSLDY | 42 |
| 79-E3H3-VL | CDR1 | RASKSISKYLA | 4 |
| | CDR2 | AGSTLQS | 10 |
| | CDR3 | QQHNEYPLT | 11 |
| 80-F10A6-VH | CDR1 | GYYMN | 43 |
| | CDR2 | EVNPITGDTTYSQNFKA | 14 |
| | CDR3 | RDYGTSLDY | 3 |
| 80-F10A6-VL | CDR1 | RASKSISRYLA | 44 |
| | CDR2 | AGSTLHL | 45 |
| | CDR3 | QQHNEYPIT | 6 |
| 90-D11B6-VH | CDR1 | GYYMN | 43 |
| | CDR2 | EINPITGGSTYNQKFVA | 46 |
| | CDR3 | RDYDTSLDY | 42 |
| 90-D11B6-VL | CDR1 | RASKSISKYLA | 4 |
| | CDR2 | AGSTLQS | 10 |
| | CDR3 | QQHNEYPLT | 11 |
| 89-E8C12-VH | CDR1 | GYYMN | 7 |
| | CDR2 | EINPITSHNTFNQKFKA | 8 |
| | CDR3 | RDYGTSLDY | 3 |
| 89-E8C12-VL | CDR1 | RASKSISKYLA | 4 |
| | CDR2 | AGSTLQS | 10 |
| | CDR3 | QQHNEYPLT | 11 |
| 85-D5A10-VH | CDR1 | GYHMN | 12 |
| | CDR2 | EINPITSDKTFNQKFKA | 13 |
| | CDR3 | RDYGTSLDY | 3 |
| 85-D5A10-VL | CDR1 | RASKSISKYLA | 4 |
| | CDR2 | AGSTLQS | 10 |
| | CDR3 | QQHNEYPIT | 6 |
| 66-D1B10-VH | CDR1 | NHWMN | 47 |
| | CDR2 | QIQLKSDNYATRYVESV | 16 |
| | CDR3 | IYYCTTGTDY | 17 |
| 66-D1B10-VL | CDR1 | KSSQSLLYSDGKTYLN | 48 |
| | CDR2 | LVSNMDS | 49 |
| | CDR3 | VQGTHFPYT | 20 |
| 63-B10B9-VH | CDR1 | GYYMN | 43 |
| | CDR2 | EINPITGGTTYNQKFRA | 50 |
| | CDR3 | RDYDTSLDY | 42 |
| 63-B10B9-VL | CDR1 | RASKSIKKYLA | 51 |
| | CDR2 | SGSTLQS | 52 |
| | CDR3 | QQHNEYPLT | 11 |
| 70-F4E5-VH | CDR1 | KHWMN | 27 |
| | CDR2 | QIQLKSDNYAIRYVDSV | 28 |
| | CDR3 | IYYCTTGTDY | 17 |
| 70-F4E5-VL | CDR1 | RSSQSLLYSNGKTYLN | 29 |
| | CDR2 | LVSNLDS | 19 |
| | CDR3 | VQGTHFPYT | 20 |
| 65-F3C7-VH | CDR1 | NHWMN | 47 |
| | CDR2 | QIQLKSDNYATRYVESV | 16 |
| | CDR3 | IYYCTTGTDY | 17 |
| 65-F3C7-VL | CDR1 | KSSQSLLYSDGKTYLN | 48 |
| | CDR2 | LVSNMDS | 49 |
| | CDR3 | VQGTHEPYT | 20 |
| 67-H2E7-VH | CDR1 | GYHMN | 12 |
| | CDR2 | EINPITSDKTENQKFKA | 13 |
| | CDR3 | RDYDTSLDY | 42 |
| 67-H2E7-VL | CDR1 | RASKSISKYLA | 4 |
| | CDR2 | AGSTLQS | 10 |
| | CDR3 | QQHNEYPLT | 11 |
| 63-C5A5-VH | CDR1 | DHWMN | 53 |
| | CDR2 | QIQLKSDNYAIRYVESV | 54 |
| | CDR3 | YYCTTGTDY | 17 |
| 63-C5A5-VL | CDR1 | KSSQSLLYSNGKTYLN | 55 |
| | CDR2 | LVSKLDS | 56 |
| | CDR3 | IQGTHFPYT | 57 |
| 69-D5G10-VH | CDR1 | NHWMN | 58 |
| | CDR2 | QIQLKSDNYATRFAESV | 59 |
| | CDR3 | YYCTTGTDY | 17 |
| 69-D5G10-VL | CDR1 | KSSQSLLYSNGKTYLN | 55 |
| | CDR2 | LVSNLDS | 19 |
| | CDR3 | IQGTHFPYT | 57 |

In some embodiments, the VH CDR1, CDR2, and CDR3 are selected from any set of VH CDR1, CDR2, and CDR3 shown in Table 1A, and the VL CDR1, CDR2, and CDR3 are selected from any set of VL CDR1, CDR2, and CDR3 shown in Table 1A. In some embodiments, the VH CDR1, CDR2, and CDR3 and the VL CDR1, CDR2, and CDR3 are selected from those derived from the same antibody in the examples.

In some embodiments, at least one, or two, or three, or four, or five, or six of the VH CDR1, CDR2, and CDR3 and the VL CDR1, CDR2, and CDR3 of the above are modified by one, two or three amino acid additions, deletions, substitutions, or the combinations thereof.

According to specific embodiments, the antibody is a humanized antibody. Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-329 (1988); and Presta, Curr. Op. Struct. Biol., 2:593-596 (1992)).

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (Jones et al., Nature, 321:522-525 (1986); Riechmann et al., Nature 332:323-327 (1988); Verhoeyen et al., Science, 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

In certain embodiments, the antibodies and the fragment thereof are humanized and comprise a heavy chain variable region comprising an amino acid sequence selected from the group consisting of SEQ ID NOs: 94-97, or a peptide having at least 90%, at least 95%, or at least 98% sequence identity to an amino acid sequence selected from the group consisting of SEQ ID NOs: 94-97.

In certain embodiments, the antibodies and the fragment thereof are humanized and comprise a light chain variable region comprising an amino acid sequence selected from the group consisting of SEQ ID NOs: 98-101 or a peptide having at least 90% %, at least 95%, or at least 98% sequence identity to an amino acid sequence selected from the group consisting of SEQ ID NOs: 98-101.

In some embodiments, the antibody is humanized but with one or more of the following back mutations on the heavy chain: 38K, 48I, 68A, 72V, and 74R, according to Kabat numbering, and combinations thereof. In some embodiments, the antibody is humanized but with one or more of the following back mutations on the light chain: 45R, 58I, and 63R according to Kabat numbering, and combinations thereof.

Non-limiting examples of heavy chain variable regions include SEQ ID NOs: 94-97. Non-limiting example of light chain variable regions include SEQ ID NOs: 98-101.

Antibodies may be produced by a process of affinity maturation in which a modified antibody is generated that has an improvement in the affinity of the antibody for antigen, compared to an unmodified parent antibody. Affinity-matured antibodies may be produced by procedures known in the art, e.g., Marks et al., Rio/Technology 10:779-783 (1992); Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154 (7): 331 0-15 9 (1995); and Hawkins et al, J. Mol. Biol. 226:889-896 (1992).

The CDR variants undergone affinity maturation are listed in Table 1B below.

TABLE 1B
Affinity matured CDR variants

| CDR | Sequence | SEQ ID NO |
|---|---|---|
| VH-CDR1 | GYHMG | 112 |
| VH-CDR2 | EINPITSDKYFNQKFKS | 113 |
| VL-CDR1 | RASASISKYLA | 114 |
| | RASKSIKKYLA | 115 |
| | RASKSISKYGA | 116 |
| VL-CDR3 | QQHNEYPII | 117 |

An example of affinity matured anti-CD24 antibody or fragment thereof includes the following CDRs: HCDR1: GYHMG (SEQ ID NO: 112), HCDR2: EINPITSDKYFNQKFKS (SEQ ID NO: 113), HCDR3: RDYGTSLDY (SEQ ID NO: 3), LCDR1: RASASISKYLA (SEQ ID NO: 114), LCDR2: AGSTLHS (SEQ ID NO: 5), and LCDR3: QQHNEYPIT (SEQ ID NO: 6).

In some embodiments, the antibody is humanized but with one or more of the following back mutations on the heavy chain: 38K, 48I, 68A, 72V, and 74R, according to Kabat numbering, and combinations thereof. In some embodiments, the antibody is humanized but with one or more of the following back mutations on the light chain: 45R, 58I, and 63R according to Kabat numbering, and combinations thereof.

Non-limiting examples of heavy chain variable regions include SEQ ID NOs: 102, 110, and 111. Non-limiting example of light chain variable regions include SEQ ID NOs: 103-109.

In some embodiments, the heavy chain variable region includes SEQ ID NO:111. In some embodiments, the light chain variable region includes SEQ ID NO:106.

In various embodiments, the present disclosure provides an antibody or antigen-binding fragment thereof having specificity to a human CD24 protein, wherein the antibody or antigen-binding fragment thereof comprises a heavy chain variable region comprising heavy chain complementarity determining regions HCDR1, HCDR2, and HCDR3, and a light chain variable region comprising light chain complementarity determining regions LCDR1, LCDR2, and LCDR3.

In one embodiments, the HCDR1 includes the amino acid sequence of SEQ ID NO:15, the HCDR2 includes the amino acid sequence of SEQ ID NO:16, the HCDR3 includes the amino acid sequence of SEQ ID NO:17, the LCDR1 includes the amino acid sequence of SEQ ID NO: 18, the LCDR2 includes the amino acid sequence of SEQ ID NO:19 and the LCDR3 includes the amino acid sequence of SEQ ID NO:20. In some embodiments, the VH comprises the amino acid sequence of SEQ ID NO:62 and the VL includes the amino acid sequence of SEQ ID NO:63.

In one embodiments, the HCDR1 includes the amino acid sequence of SEQ ID NO:21, the HCDR2 includes the amino acid sequence of SEQ ID NO:22, the HCDR3 includes the amino acid sequence of SEQ ID NO:23, the LCDR1 includes the amino acid sequence of SEQ ID NO: 24, the LCDR2 includes the amino acid sequence of SEQ ID NO:25 and the LCDR3 includes the amino acid sequence of SEQ ID NO:26. In some embodiments, the VH comprises the amino acid sequence of SEQ ID NO:64 and the VL includes the amino acid sequence of SEQ ID NO:65.

In one embodiments, the HCDR1 includes the amino acid sequence of SEQ ID NO:27, the HCDR2 includes the amino acid sequence of SEQ ID NO:28, the HCDR3 includes the amino acid sequence of SEQ ID NO:17, the LCDR1 includes the amino acid sequence of SEQ ID NO: 29, the LCDR2 includes the amino acid sequence of SEQ ID NO:30 and the LCDR3 includes the amino acid sequence of SEQ ID NO:20. In some embodiments, the VH comprises the amino acid sequence of SEQ ID NO:66 and the VL includes the amino acid sequence of SEQ ID NO:67.

In one embodiments, the HCDR1 includes the amino acid sequence of SEQ ID NO:31, the HCDR2 includes the amino acid sequence of SEQ ID NO:32, the HCDR3 includes the amino acid sequence of SEQ ID NO:33, the LCDR1 includes the amino acid sequence of SEQ ID NO: 34, the LCDR2 includes the amino acid sequence of SEQ ID NO:25 and the LCDR3 includes the amino acid sequence of SEQ ID NO:35. In some embodiments, the VH comprises the amino acid sequence of SEQ ID NO:68 and the VL includes the amino acid sequence of SEQ ID NO:69.

In one embodiments, the HCDR1 includes the amino acid sequence of SEQ ID NO:36, the HCDR2 includes the amino acid sequence of SEQ ID NO:37, the HCDR3 includes the amino acid sequence of SEQ ID NO:38, the LCDR1 includes the amino acid sequence of SEQ ID NO: 39, the LCDR2 includes the amino acid sequence of SEQ ID NO:40 and the LCDR3 includes the amino acid sequence of SEQ ID NO:41. In some embodiments, the VH comprises the amino acid sequence of SEQ ID NO:70 and the VL includes the amino acid sequence of SEQ ID NO:71.

In one embodiments, the HCDR1 includes the amino acid sequence of SEQ ID NO:12, the HCDR2 includes the amino acid sequence of SEQ ID NO:13, the HCDR3 includes the amino acid sequence of SEQ ID NO:42, the LCDR1 includes the amino acid sequence of SEQ ID NO: 4, the LCDR2 includes the amino acid sequence of SEQ ID NO:10 and the LCDR3 includes the amino acid sequence of SEQ ID NO:11. In some embodiments, the VH comprises the amino acid sequence of SEQ ID NO:72 and the VL includes the amino acid sequence of SEQ ID NO:73.

In one embodiments, the HCDR1 includes the amino acid sequence of SEQ ID NO:43, the HCDR2 includes the amino acid sequence of SEQ ID NO:14, the HCDR3 includes the amino acid sequence of SEQ ID NO:3, the LCDR1 includes the amino acid sequence of SEQ ID NO: 44, the LCDR2 includes the amino acid sequence of SEQ ID NO:45 and the LCDR3 includes the amino acid sequence of SEQ ID NO:6. In some embodiments, the VH comprises the amino acid sequence of SEQ ID NO:74 and the VL includes the amino acid sequence of SEQ ID NO:75.

In one embodiments, the HCDR1 includes the amino acid sequence of SEQ ID NO:43, the HCDR2 includes the amino acid sequence of SEQ ID NO:46, the HCDR3 includes the amino acid sequence of SEQ ID NO:42, the LCDR1 includes the amino acid sequence of SEQ ID NO: 4, the LCDR2 includes the amino acid sequence of SEQ ID NO:10 and the LCDR3 includes the amino acid sequence of SEQ ID NO:11. In some embodiments, the VH comprises the amino acid sequence of SEQ ID NO:76 and the VL includes the amino acid sequence of SEQ ID NO:77.

In one embodiments, the HCDR1 includes the amino acid sequence of SEQ ID NO:7, the HCDR2 includes the amino acid sequence of SEQ ID NO:8, the HCDR3 includes the amino acid sequence of SEQ ID NO:3, the LCDR1 includes the amino acid sequence of SEQ ID NO: 4, the LCDR2 includes the amino acid sequence of SEQ ID NO:10 and the LCDR3 includes the amino acid sequence of SEQ ID NO:11. In some embodiments, the VH comprises the amino acid sequence of SEQ ID NO:78 and the VL includes the amino acid sequence of SEQ ID NO:79.

In one embodiments, the HCDR1 includes the amino acid sequence of SEQ ID NO:12, the HCDR2 includes the amino acid sequence of SEQ ID NO:13, the HCDR3 includes the amino acid sequence of SEQ ID NO:3, the LCDR1 includes the amino acid sequence of SEQ ID NO: 4, the LCDR2 includes the amino acid sequence of SEQ ID NO:10 and the LCDR3 includes the amino acid sequence of SEQ ID NO:6. In some embodiments, the VH comprises the amino acid sequence of SEQ ID NO:80 and the VL includes the amino acid sequence of SEQ ID NO:81.

In one embodiments, the HCDR1 includes the amino acid sequence of SEQ ID NO:47, the HCDR2 includes the amino acid sequence of SEQ ID NO:16, the HCDR3 includes the amino acid sequence of SEQ ID NO:17, the LCDR1 includes the amino acid sequence of SEQ ID NO: 48, the LCDR2 includes the amino acid sequence of SEQ ID NO:49 and the LCDR3 includes the amino acid sequence of SEQ ID NO:20. In some embodiments, the VH comprises the amino acid sequence of SEQ ID NO:82 and the VL includes the amino acid sequence of SEQ ID NO:83.

In one embodiments, the HCDR1 includes the amino acid sequence of SEQ ID NO:43, the HCDR2 includes the amino acid sequence of SEQ ID NO:50, the HCDR3 includes the amino acid sequence of SEQ ID NO:42, the LCDR1 includes the amino acid sequence of SEQ ID NO: 51, the LCDR2 includes the amino acid sequence of SEQ ID NO:52 and the LCDR3 includes the amino acid sequence of SEQ ID NO:11. In some embodiments, the VH comprises the amino acid sequence of SEQ ID NO:84 and the VL includes the amino acid sequence of SEQ ID NO:85.

In one embodiments, the HCDR1 includes the amino acid sequence of SEQ ID NO:27, the HCDR2 includes the amino acid sequence of SEQ ID NO:28, the HCDR3 includes the amino acid sequence of SEQ ID NO:17, the LCDR1 includes the amino acid sequence of SEQ ID NO: 29, the LCDR2 includes the amino acid sequence of SEQ ID NO:19 and the LCDR3 includes the amino acid sequence of SEQ ID NO:20. In some embodiments, the VH comprises the amino acid sequence of SEQ ID NO:66 and the VL includes the amino acid sequence of SEQ ID NO:86.

In one embodiments, the HCDR1 includes the amino acid sequence of SEQ ID NO:47, the HCDR2 includes the amino acid sequence of SEQ ID NO:16, the HCDR3 includes the amino acid sequence of SEQ ID NO:17, the LCDR1 includes the amino acid sequence of SEQ ID NO: 48, the LCDR2 includes the amino acid sequence of SEQ ID NO:49 and the LCDR3 includes the amino acid sequence of SEQ ID NO:20. In some embodiments, the VH comprises the amino acid sequence of SEQ ID NO:87 and the VL includes the amino acid sequence of SEQ ID NO:83.

In one embodiments, the HCDR1 includes the amino acid sequence of SEQ ID NO:12, the HCDR2 includes the amino acid sequence of SEQ ID NO:13, the HCDR3 includes the amino acid sequence of SEQ ID NO:42, the LCDR1 includes the amino acid sequence of SEQ ID NO: 4, the LCDR2 includes the amino acid sequence of SEQ ID NO:10 and the LCDR3 includes the amino acid sequence of SEQ ID NO:11. In some embodiments, the VH comprises the amino acid sequence of SEQ ID NO:88 and the VL includes the amino acid sequence of SEQ ID NO:89.

In one embodiments, the HCDR1 includes the amino acid sequence of SEQ ID NO:53, the HCDR2 includes the amino acid sequence of SEQ ID NO:54, the HCDR3 includes the amino acid sequence of SEQ ID NO:17, the LCDR1 includes the amino acid sequence of SEQ ID NO: 55, the LCDR2 includes the amino acid sequence of SEQ ID NO:56 and the LCDR3 includes the amino acid sequence of SEQ ID NO:57. In some embodiments, the VH comprises the amino acid sequence of SEQ ID NO:90 and the VL includes the amino acid sequence of SEQ ID NO:91.

In one embodiments, the HCDR1 includes the amino acid sequence of SEQ ID NO:58, the HCDR2 includes the amino acid sequence of SEQ ID NO:59, the HCDR3 includes the amino acid sequence of SEQ ID NO:17, the LCDR1 includes the amino acid sequence of SEQ ID NO: 55, the LCDR2 includes the amino acid sequence of SEQ ID NO:19 and the LCDR3 includes the amino acid sequence of SEQ ID NO:57. In some embodiments, the VH comprises the amino acid sequence of SEQ ID NO:92 and the VL includes the amino acid sequence of SEQ ID NO:93.

The CDRs, heavy chain variable regions and light chain variable regions of the present disclosure can be further modified. In some embodiments, the modified heavy chain variable region or light chain variable region retains at least about 70%, 75%, 80%, 85%, 90%, 95%, 98% or 99% sequence identity and is still capable of binding to CD24.

In some embodiments, the modification is substitution at no more than one hot spot position from each of the CDRs. In some embodiments, the modification is substitution at one, two or three such hot spot positions. In one embodiment, the modification is substitution at one of the hot spot positions. Such substitutions, in some embodiments, are conservative substitutions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Non-limiting examples of conservative amino acid substitutions are provided in the table below, where a similarity score of 0 or higher indicates conservative substitution between the two amino acids.

### Amino Acid Similarity Matrix

| | C | G | P | S | A | T | D | E | N | Q | H | K | R | V | M | I | L | F | Y | W |
|---|---|---|---|---|---|---|---|---|---|---|---|---|---|---|---|---|---|---|---|---|
| W | −8 | −7 | −6 | −2 | −6 | −5 | −7 | −7 | −4 | −5 | −3 | −3 | 2 | −6 | −4 | −5 | −2 | 0 | 0 | 17 |
| Y | 0 | −5 | −5 | −3 | −3 | −3 | −4 | −4 | −2 | −4 | 0 | −4 | −5 | −2 | −2 | −1 | −1 | 7 | 10 | |
| F | −4 | −5 | −5 | −3 | −4 | −3 | −6 | −5 | −4 | −5 | −2 | −5 | −4 | −1 | 0 | 1 | 2 | 9 | | |
| L | −6 | −4 | −3 | −3 | −2 | −2 | −4 | −3 | −3 | −2 | −2 | −3 | −3 | 2 | 4 | 2 | 6 | | | |
| I | −2 | −3 | −2 | −1 | −1 | 0 | −2 | −2 | −2 | −2 | −2 | −2 | −2 | 4 | 2 | 5 | | | | |
| M | −5 | −3 | −2 | −2 | −1 | −1 | −3 | −2 | 0 | −1 | −2 | 0 | 0 | 2 | 6 | | | | | |
| V | −2 | −1 | −1 | −1 | 0 | 0 | −2 | −2 | −2 | −2 | −2 | −2 | −2 | 4 | | | | | | |
| R | −4 | −3 | 0 | 0 | −2 | −1 | −1 | −1 | 0 | 1 | 2 | 3 | 6 | | | | | | | |
| K | −5 | −2 | −1 | 0 | −1 | 0 | 0 | 0 | 1 | 1 | 0 | 5 | | | | | | | | |
| H | −3 | −2 | 0 | −1 | −1 | −1 | 1 | 1 | 2 | 3 | 6 | | | | | | | | | |
| Q | −5 | −1 | 0 | −1 | 0 | −1 | 2 | 2 | 1 | 4 | | | | | | | | | | |
| N | −4 | 0 | −1 | 1 | 0 | 0 | 2 | 1 | 2 | | | | | | | | | | | |
| E | −5 | 0 | −1 | 0 | 0 | 0 | 3 | 4 | | | | | | | | | | | | |
| D | −5 | 1 | −1 | 0 | 0 | 0 | 4 | | | | | | | | | | | | | |
| T | −2 | 0 | 0 | 1 | 1 | 3 | | | | | | | | | | | | | | |
| A | −2 | 1 | 1 | 1 | 2 | | | | | | | | | | | | | | | |
| S | 0 | 1 | 1 | 1 | | | | | | | | | | | | | | | | |
| P | −3 | −1 | 6 | | | | | | | | | | | | | | | | | |
| G | −3 | 5 | | | | | | | | | | | | | | | | | | |
| C | 12 | | | | | | | | | | | | | | | | | | | |

### Conservative Amino Acid Substitutions

| For Amino | Substitution With |
|---|---|
| Alanine | D-Ala, Gly, Aib, β-Ala, L-Cys, D-Cys |
| Arginine | D-Arg, Lys, D-Lys, Orn D-Orn |
| Asparagine | D-Asn, Asp, D-Asp, Glu, D-Glu Gln, D-Gln |
| Aspartic Acid | D-Asp, D-Asn, Asn, Glu, D-Glu, Gln, D-Gln |
| Cysteine | D-Cys, S-Me-Cys, Met, D-Met, |
| | Thr, D-Thr, L-Ser, D-Ser |
| Glutamine | D-Gln, Asn, D-Asn, Glu, D-Glu, Asp, D-Asp |
| Glutamic Acid | D-Glu, D-Asp, Asp, Asn, D-Asn, Gln, D-Gln |
| Glycine | Ala, D-Ala, Pro, D-Pro, Aib, β-Ala |
| Isoleucine | D-Ile, Val, D-Val, Leu, D-Leu, Met, D-Met |
| Leucine | Val, D-Val, Met, D-Met, D-Ile, D-Leu, Ile |
| Lysine | D-Lys, Arg, D-Arg, Orn, D-Orn |
| Methionine | D-Met, S-Me-Cys, Ile, D-Ile, |
| | Leu, D-Leu, Val, D-Val |
| Phenylalanine | D-Phe, Tyr, D-Tyr, His, D-His, Trp, D-Trp |
| Proline | D-Pro |
| Serine | D-Ser, Thr, D-Thr, allo-Thr, L-Cys, D-Cys |
| Threonine | D-Thr, Ser, D-Ser, allo-Thr, |
| | Met, D-Met, Val, D-Val |
| Tyrosine | D-Tyr, Phe, D-Phe, His, D-His, Trp, D-Trp |
| Valine | D-Val, Leu, D-Leu, Ile, D-Ile, Met, D-Met |

It will also be understood by one of ordinary skill in the art that antibodies as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, 95%, 98%, or 99% identical to the starting sequence.

In certain embodiments, the antibody provided herein further comprises a heavy chain constant region, a light chain constant region, an Fc region, or the combination thereof.

The Fc region can be engineered to enhance effector function. IgG antibodies can induce direct anti-tumor effects by way of indirect anti-tumor effects via the Fc-mediated effector functions that engage other immune cells or killer mechanisms. “Effector functions” or “antibody effector functions” as used herein refer to biological activities attributable to the binding of Fc region of an antibody to its effectors such as C1 complex and Fc receptor (FcγRIIa or FcγRIIIa). Exemplary effector functions include: complement dependent cytotoxicity (CDC) induced by interaction of antibodies and C1q on the C1 complex; antibody-dependent cell-mediated cytotoxicity (ADCC) induced by binding of Fc region of an antibody to Fc receptor on an effector cell; and antibody dependent cell mediated phagocytosis (ADCP), where nonspecific cytotoxic cells that express FcγRs recognize bound antibody on a target cell and subsequently cause phagocytosis of the target cell. Among the four IgG subclasses, IgG1 and IgG3 induce the strongest Fc-effector functions. However, since IgG1 has the longest half-life and is more stable than IgG3, most therapeutic antibodies with Fc-mediated functions are of IgG1 isotype.

In certain embodiments, the Fc region of the antibodies provided herein is engineered to enhance the effector function, such as ADCC or CDC. Various methods (mainly Fc mutations) can be found to enhance the Fc-mediated effector function, such as those described in the PCT publications WO2007024249A2 and WO2011044368A1, which are incorporated herein by reference in their entireties. In certain embodiments, the Fc region is engineered to include S298A/E333A/K334A mutations in human IgG1. Such Fc mutation combination is reported to enhance binding to FcγRIIIa, thus enhancing ADCC (See Shields R. L. et al, J. Biol. Chem 276:6591-6604 (2001)).

In one aspect, the present disclosure provides a multispecific antibody or fragment thereof. In certain embodiments, the present disclosure provides a bifunctional molecule, comprising a first antigen-binding portion having specificity to a human CD24 protein and a second portion having specificity to a second protein, wherein the first antigen-binding portion comprises an anti-CD24 antibody or fragment thereof as provided herein.

In certain embodiments, the second portion is an antibody or an antigen binding fragment thereof. In certain embodiments, the second portion has specificity to immune checkpoints, such as PD-1, PD-L1, CTLA-4, TIM-3 or LAG-3. In certain embodiments, the second portion has specificity to other tumor antigen, including but not limited to CD3, EphA2, CA-125, gangliosides G (D2), G (M2) and G (D3), CD20, CD52, CD33, Ep-CAM, CEA, bombesin-like peptides, PSA, HER2/neu, epidermal growth factor receptor (EGFR), erbB2, erbB3/HER3, erbB4, CD44v6, Ki-67, cancer-associated mucin, VEGF, VEGFRs (e.g., VEGFR3), estrogen receptors, Lewis-Y antigen, TGFβ1, IGF-1 receptor, EGFα, c-Kit receptor, transferrin receptor, IL-2R or CO17-1A.

In certain embodiments, the antibody comprises an amino acid sequence or one or more moieties not normally associated with an antibody. Exemplary modifications are described in more detail below. For example, an antibody of the disclosure may comprise a flexible linker sequence, or may be modified to add a functional moiety (e.g., PEG, a drug, a toxin, or a label).

Antibodies, variants, or derivatives thereof of the disclosure include derivatives that are modified, i.e., by the covalent attachment of any type of molecule to the antibody such that covalent attachment does not prevent the antibody from binding to the epitope. For example, but not by way of limitation, the antibodies can be modified, e.g., by glycosylation, acetylation, pegylation, phosphorylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the antibodies may contain one or more non-classical amino acids.

In some embodiments, the antibodies may be conjugated to therapeutic agents, prodrugs, peptides, proteins, enzymes, viruses, lipids, biological response modifiers, pharmaceutical agents, or PEG.

The antibodies may be conjugated or fused to a therapeutic agent, which may include detectable labels such as radioactive labels, an immunomodulator, a hormone, an enzyme, an oligonucleotide, a photoactive therapeutic or diagnostic agent, a cytotoxic agent, which may be a drug or a toxin, an ultrasound enhancing agent, a non-radioactive label, a combination thereof and other such agents known in the art.

### Polynucleotides Encoding the Antibodies and Methods of Preparing the Antibodies

The present disclosure also provides isolated polynucleotides or nucleic acid molecules encoding the antibodies, variants or derivatives thereof of the disclosure. The polynucleotides of the present disclosure may encode the entire heavy and light chain variable regions of the antigen-binding polypeptides, variants or derivatives thereof on the same polynucleotide molecule or on separate polynucleotide molecules. Additionally, the polynucleotides of the present disclosure may encode portions of the heavy and light chain variable regions of the antigen-binding polypeptides, variants or derivatives thereof on the same polynucleotide molecule or on separate polynucleotide molecules.

Methods of making antibodies are well known in the art and described herein. In certain embodiments, both the variable and constant regions of the antigen-binding polypeptides of the present disclosure are fully human. Fully human antibodies can be made using techniques described in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140 which are incorporated by reference in their entireties.

### Treatment and Diagnostic Methods

As described herein, the antibodies, variants or derivatives of the present disclosure may be used in certain treatment and diagnostic methods.

The present disclosure is further directed to antibody-based therapies which involve administering the antibodies of the disclosure to a patient such as an animal, a mammal, and a human for treating one or more of the disorders or conditions described herein. Therapeutic compounds of the disclosure include, but are not limited to, antibodies of the disclosure (including variants and derivatives thereof as described herein) and nucleic acids or polynucleotides encoding antibodies of the disclosure (including variants and derivatives thereof as described herein).

In some embodiments, provided are methods for treating a cancer in a patient in need thereof. The method, in one embodiment, entails administering to the patient an effective amount of an antibody or antigen-binding fragment thereof of the present disclosure.

In some embodiments, provided are uses of the antibodies or antigen-binding fragments thereof of the present disclosure in the manufacture of a medicament for treating a cancer in a patient in need thereof.

In some embodiments, provided are the antibodies or antigen-binding fragments thereof of the present disclosure for use in the treatment of a cancer in a patient in need thereof.

Non-limiting examples of cancers include bladder cancer, breast cancer, colorectal cancer, endometrial cancer, esophageal cancer, head and neck cancer, kidney cancer, leukemia, liver cancer, lung cancer, lymphoma, melanoma, pancreatic cancer, prostate cancer, and thyroid cancer.

Additional diseases or conditions associated with increased cell survival, that may be treated, prevented, diagnosed and/or prognosed with the antibodies or variants, or derivatives thereof of the disclosure include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

As demonstrated in the experimental examples, the antibodies of the present disclosure can activate immune response which can then be useful for treating infections.

Infection is the invasion of an organism's body tissues by disease-causing agents, their multiplication, and the reaction of host tissues to these organisms and the toxins they produce. An infection can be caused by infectious agents such as viruses, viroids, prions, bacteria, nematodes such as parasitic roundworms and pinworms, arthropods such as ticks, mites, fleas, and lice, fungi such as ringworm, and other macroparasites such as tapeworms and other helminths. In one aspect, the infectious agent is a bacterium, such as Gram negative bacterium. In one aspect, the infectious agent is virus, such as DNA viruses, RNA viruses, and reverse transcribing viruses. Non-limiting examples of viruses include Adenovirus, Coxsackievirus, Epstein-Barr virus, Hepatitis A virus, Hepatitis B virus, Hepatitis C virus, Herpes simplex virus, type 1, Herpes simplex virus, type 2, Cytomegalovirus, Human herpesvirus, type 8, HIV, Influenza virus, Measles virus, Mumps virus, Human papillomavirus, Parainfluenza virus, Poliovirus, Rabies virus, Respiratory syncytial virus, Rubella virus, Varicella-zoster virus.

The antibodies of the present disclosure can also be used to treat an infectious disease caused by a microorganism, or kill a microorganism, by targeting the microorganism and an immune cell to effect elimination of the microorganism. In one aspect, the microorganism is a virus including RNA and DNA viruses, a Gram positive bacterium, a Gram negative bacterium, a protozoa or a fungus.

In another embodiment, the antibodies and antigen binding fragments of the present disclosure can be used for treating inflammatory diseases or conditions, and autoimmune diseases or conditions.

In some embodiments, the inflammatory disease or condition to be treated by the disclosed antibodies, fragments and compositions includes one or more of Alzheimer's disease, Addison's disease, atherosclerosis, ankylosing spondylitis, arthritis, osteoarthritis (OA), rheumatoid arthritis (RA), psoriatic arthritis (PA), ankylosing spondylitis, asthma, atherosclerosis, chronic obstructive pulmonary disease (COPD), Crohn's disease, colitis, dermatitis, diverticulitis, fibromyalgia, hepatitis, irritable bowel syndrome (IBS), systemic lupus erythematous (SLE), nephritis, Parkinson's disease (PD), vasculitis, and ulcerative colitis.

In some embodiments, the autoimmune disease or condition to be treated by the disclosed antibodies, fragments and compositions includes one or more of alopecia areata, autoimmune hemolytic anemia, autoimmune hepatitis, dermatomyositis, diabetes (type 1), celiac disease, autoimmune juvenile idiopathic arthritis, glomerulonephritis, Graves' disease, Guillain-Barré syndrome, idiopathic thrombocytopenia purpura, myasthenia gravis, autoimmune myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus erythematosus, autoimmune thyroiditis, Hashimoto's thyroiditis, autoimmune uveitis, vitiligo, and granulomatosis with polyangiitis (Wegener's).

Rheumatoid arthritis (RA) is a long-term autoimmune disorder that primarily affects joints. It typically results in warm, swollen, and painful joints. Pain and stiffness often worsen following rest. Most commonly, the wrist and hands are involved, with the same joints typically involved on both sides of the body. The disease may also affect other parts of the body. While the cause of rheumatoid arthritis is not clear, it is believed to involve a combination of genetic and environmental factors. The underlying mechanism involves the body's immune system attacking the joints. This results in inflammation and thickening of the joint capsule. The goals of treatment are to reduce pain, decrease inflammation, and improve a person's overall functioning. Pain medications, steroids, and NSAIDs are frequently used to help with symptoms. A group of medications called disease-modifying antirheumatic drugs (DMARDs), such as hydroxychloroquine and methotrexate, may be used to try to slow the progression of disease.

Osteoarthritis (OA) is a type of joint disease that results from breakdown of joint cartilage and underlying bone. The most common symptoms are joint pain and stiffness. Initially, symptoms may occur only following exercise, but over time may become constant. Other symptoms may include joint swelling, decreased range of motion, and when the back is affected weakness or numbness of the arms and legs. Causes include previous joint injury, abnormal joint or limb development, and inherited factors. Risk is greater in those who are overweight, have one leg of a different length, and have jobs that result in high levels of joint stress. Osteoarthritis is believed to be caused by mechanical stress on the joint and low grade inflammatory processes. Treatment includes exercise, efforts to decrease joint stress, support groups, and pain medications.

Multiple sclerosis (MS) is a demyelinating disease in which the insulating covers of nerve cells in the brain and spinal cord are damaged. This damage disrupts the ability of parts of the nervous system to communicate, resulting in a range of signs and symptoms, including physical, mental, and sometimes psychiatric problems. Specific symptoms can include double vision, blindness in one eye, muscle weakness, trouble with sensation, or trouble with coordination. While the cause is not clear, the underlying mechanism is thought to be either destruction by the immune system or failure of the myelin-producing cells. There is no known cure for multiple sclerosis. Treatments attempt to improve function after an attack and prevent new attacks.

Asthma is a common long-term inflammatory disease of the airways of the lungs. It is characterized by variable and recurring symptoms, reversible airflow obstruction, and bronchospasm. Symptoms include episodes of wheezing, coughing, chest tightness, and shortness of breath. Asthma is thought to be caused by a combination of genetic and environmental factors. Environmental factors include exposure to air pollution and allergens. Asthma is classified according to the frequency of symptoms, forced expiratory volume in one second (FEV1), and peak expiratory flow rate. It may also be classified as atopic or non-atopic, where atopy refers to a predisposition toward developing a type 1 hypersensitivity reaction. There is no cure for asthma. Symptoms can be prevented by avoiding triggers, such as allergens and irritants, and by the use of inhaled corticosteroids. Long-acting beta agonists (LABA) or antileukotriene agents may be used in addition to inhaled corticosteroids if asthma symptoms remain uncontrolled. Treatment of rapidly worsening symptoms is usually with an inhaled short-acting beta-2 agonist such as salbutamol and corticosteroids taken by mouth. In very severe cases, intravenous corticosteroids, magnesium sulfate, and hospitalization may be required.

Chronic obstructive pulmonary disease (COPD) is a type of obstructive lung disease characterized by long-term poor airflow. COPD can include two main conditions, emphysema and chronic bronchitis. In emphysema, the walls between many of the air sacs are damaged. As a result, the air sacs lose their shape and become floppy. This damage also can destroy the walls of the air sacs, leading to fewer and larger air sacs instead of many tiny ones. If this happens, the amount of gas exchange in the lungs is reduced. In chronic bronchitis, the lining of the airways stays constantly irritated and inflamed, and this causes the lining to swell. Lots of thick mucus forms in the airways, making it hard to breathe. There is no known cure for COPD, but the symptoms are treatable and its progression can be delayed.

Pain is a distressing feeling often caused by intense or damaging stimuli, such as stubbing a toe, burning a finger, putting alcohol on a cut, or bumping the “funny bone”. Pain is a complex, subjective phenomenon, defining pain has been a challenge. Pain is also referred to as an unpleasant sensory and emotional experience associated with actual or potential tissue damage. Pain is sometimes regarded as a symptom of an underlying condition, such as inflammation.

A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

Methods of administration of the antibodies, variants or include but are not limited to intradermal, intramuscular, intraperitoneal, intravenous, subcutaneous, intranasal, epidural, and oral routes. The antigen-binding polypeptides or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the antigen-binding polypeptides of the disclosure may be administered orally, rectally, parenterally, intracistemally, intravaginally, intraperitoneally, topically (as by powders, ointments, drops or transdermal patch), bucally, or as an oral or nasal spray.

The term “parenteral” as used herein refers to modes of administration which include intravenous, intramuscular, intraperitoneal, intrasternal, subcutaneous and intra-articular injection and infusion.

Administration can be systemic or local. In addition, it may be desirable to introduce the antibodies of the disclosure into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

It may be desirable to administer the antigen-binding polypeptides or compositions of the disclosure locally to the area in need of treatment; this may be achieved by, for example, and not by way of limitation, local infusion during surgery, topical application, e.g., in conjunction, with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the disclosure, care must be taken to use materials to which the protein does not absorb.

Methods of detecting expression of a human CD24 protein in a sample are also provided, in some embodiments, comprising contacting the sample with the antibody or fragment thereof, and detecting the binding which indicates expression of CD24 in the sample.

In certain embodiments, provided are uses of the antibodies or antigen-binding fragments thereof of the present disclosure in the manufacture of a kit for detecting expression of a human CD24 protein in a sample.

### Compositions

The present disclosure also provides pharmaceutical compositions. Such compositions comprise an effective amount of an antibody, and an acceptable carrier. In some embodiments, the composition further includes a second anticancer agent (e.g., an immune checkpoint inhibitor).

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### EXAMPLES

### Example 1. Generation of Mouse Monoclonal Antibodies Against Human CD24

This example shows the generation of anti-human-CD24 mouse monoclonal antibodies using the hybridoma technology.

### Immunization

Recombinant human CD24 proteins containing the 33 core amino acids were used as the immunogen to raise anti-human CD24 antibodies. Mice or SD rats were immunized with immunogen and immune response was monitored. Plasma was screened by ELISA binding assay. The mice with high titers of anti-CD24 immunoglobulin were selected for fusion and further screening.

### Fusion and Hybridoma Screening

The mouse splenocytes, isolated from the mice, were fused with a mouse myeloma cell line based upon standard protocols. Single cell suspensions of splenic lymphocytes from immunized mice were fused to one-third the number of SP2/0 non secreting mouse myeloma cells with electrofusion machine. Individual wells of hybridoma were then screened by ELISA for mouse anti-CD24 monoclonal IgG antibodies. The stable sub clones were then cultured in vitro to generate small amount of antibody in tissue culture medium for further characterization with various functional assays.

Clones showing strong blocking ability in CD24-responsive reporter assay were selected for sub cloning. After the screenings, 18 clones (74-H4C6 (also named 4451), 76-F8C6, 59-G9D7, 75-H8B9, 62-F7E9, 45-H1E4, 79-E3H3, 80-F10A6, 90-D11B6, 89-E8C12 (also named 4428), 85-D5A10 (also named 4434), 66-D1B10, 63-B10B9, 70-F4E5, 65-F3C7, 67-H2E7, 63-C5A5, and 69-D5G10) were selected. Sequences of these clones are list in Table 2. Chimeric antibodies fused to human Kappa light chain constant region and IgG1 or human IgG4 Fc of these hybridoma were generated for further characterization.

TABLE 2
Antibody sequences selected from screening

| Variable | | |
|---|---|---|
| region | Sequences* (CDR underlined and bold) | SEQ ID NO: |
| 74-H4C6-VH | MGWSCIILFLVATATGVHSEVQLQQSGPELVKPGASVKISCKASG | 60 |
| | YDFTGYHMNWVKQSPEKSLEWIGEINPITSDKTFNQKFKSKATLT | |
| | VDRSSSTAYMYLKSLTSEDSAVYYCARRDYGTSLDYWGQGTTLTV | |
| | SS | |
| 74-H4C6-VL | MGWSCIILFLVATATGVHSDVQITQSPSYLAASPGETITIYCRAS | 61 |
| | KSISKYLAWYQEKPEKTNRLLIYAGSTLHSGIPSRFRGSGSGKDF | |
| | TLTVSGLEPEDFAMYYCQQHNEYPITFGAGTKLELK | |
| 76-F8C6-VH | MGWSCIILFLVATATGVHSEVKLEESGGGLVQPGGSMKLSCVASG | 62 |
| | FTFTNSWMNWVRQSPERGLDWVAQIQLKSDNYATRYVESVKGRFT | |
| | ISRDDSKNSVYLQMNNLRAEDTGIYYCTTGTDYWSQGTTLTVSS | |
| 76-F8C6-VL | MGWSCIILFLVATATGVHSDVVVTQTPLTLSVTIGQTASISCKSS | 63 |
| | QSLLFSNGKTYLNWLFQRPGQSPKRLIYLVSNLDSGVPDRFTGSG | |
| | SGTDFTLKISRVEAEDLGVYYCVQGTHEPYTFGGGTKLEIK | |
| 59-G9D7-VH | MGWSCIILFLVATATGVHSQVQLQQPGADLVKPGASVKLSCKASG | 64 |
| | YTVTTYWMHWVRQRPGRGLEWIGRIDPNSGETKYSEKFKNKATLT | |
| | VDRPSSTAYMQLSSLTFEDSAVYFCAIYYYLNNFLFDYWGQGTTL | |
| | TVSS | |
| 59-G9D7-VL | MGWSCIILFLVATATGVHSENVLTQSPAIMSASPGEKVTMTCRAS | 65 |
| | SSVSSSYLHWYQKKSGASPKLWIYSTSNLASGVPARFSGSGSGTS | |
| | YSLTISSVEAEDAATYYCQQYSGYPLTFGAGTKLELK | |
| 75-H8B9-VH | MGWSCIILFLVATATGVHSEVNLEESGGGLVQPGGSRQLSCVASG | 66 |
| | LTFSKHWMNWVRQSPDKGLEWVAQIQLKSDNYAIRYVDSVKGRFT | |
| | ISRDDSENTVYLQMNNLRAEDTGIYYCTTGTDYWGQGTTLTVSS | |
| 75-H8B9-VL | MGWSCIILFLVATATGVHSDVVMTQTPLTLSVTIGQPASISCRSS | 67 |
| | QSLLYSNGKTYLNWLLQRPGQSPKRLIYVVSNLDSGVPDRFIGSG | |
| | SGTDFTLKISRVEAEDLGVYYCVQGTHFPYTFGGGTKLEIK | |
| 62-F7E9-VH | MGWSCIILFLVATATGVHSQVQLQQPGAELVKPGASVKLSCKASG | 68 |
| | YTSTSYWLHWVKQRPGRGLEWIGRIDPNSGETKFNEKFKSQATLT | |
| | VDKPSSTAYMQLNSLTSEDSAVYYCALYYYGSILFDYWGQGTTLT | |
| | VSS | |
| 62-F7E9-VL | MGWSCIILFLVATATGVHSENVLTQSPAIMSASPGEKVTMTCRTS | 69 |
| | SIISSGFLHWYQQKSGASPKLWIYSTSNLASGVPARFSGSGSGTS | |
| | YSLTISSVEAEDAATYYCQQYSGHPLTFGAGTKLELK | |
| 45-H1E4-VH | MGWSCIILFLVATATGVHSQIQLVQSGPELKKPGETVKISCKASG | 70 |
| | YTFTTYGMSWMKQAPGKGLKWMGWINTYSGVPAYADDFKGRFAFS | |
| | LDTSASTAYLQINNLKNEDTATYFCARSGYPWSPHSYSTDYWGQG | |
| | ISVTVSS | |
| 45-H1E4-VL | MGWSCIILFLVATATGVHSDVLLTQTPLSLPVSLGDQASISCKSS | 71 |
| | QSIVHSNGNTYLEWYLQKPGQSPKRLIYKVSNRFSGVPDRFSGSG | |
| | SGTDFTLKISRVEAEDLGVYYCFQGSHVPWTFGGGTKLEIK | |
| 79-E3H3-VH | MGWSCIILFLVATATGVHSEVQLQQSGPELVKPGASVKISCKASG | 72 |
| | YEFTGYHMNWVKQSPEKSLEWIGEINPITSDKTFNQKFKAKATLT | |
| | VDRSSSTAYMHLKSLTSEDSAVYYCARRDYDTSLDYWGQGTTLTV | |
| | SS | |
| 79-E3H3-VL | MGWSCIILFLVATATGVHSDVQITQSPSYLAASPGETITIYCRAS | 73 |
| | KSISKYLAWYQEKPEKTNRLLIYAGSTLQSGIPSRFRGSGSGKDF | |
| | TLTVSGLEPEDFAMYYCQQHNEYPLTFGAGTKLELK | |
| 80-F10A6- | MGWSCIILFLVATATGVHSEVQLQQSGPVLVNPGASVKISCKASG | 74 |
| VH | YSFTGYYMNWVKQSPEKSLEWIGEVNPITGDTTYSQNFKAKTTLT | |
| | VDRSSSTAYMQLKSLTSEDSAVYYCTRRDYGTSLDYWGQGTTLTV | |
| | SS | |
| 80-F10A6-VL | MGWSCIILFLVATATGVHSDVQITQSPSYLAASPGETITIHCRAS | 75 |
| | KSISRYLAWYQEKPGKINTLLIYAGSTLHLGIPSRFSGSGSGTDF | |
| | TLTISSLELEDFAMYYCQQHNEYPITFGAGTKLELK | |
| 90-D11B6- | MGWSCIILFLVATATGVHSEVQLQQSGPELVKPGASVRISCKASG | 76 |
| VH | YSFTGYYMNWVKQSPEKNLEWIGEINPITGGSTYNQKFVAKATLT | |
| | VDRSSSTAYMQLKSLTSEDSAVYYCTRRDYDTSLDYWGQGTTLTV | |
| | SS | |
| 90-D11B6- | MGWSCIILFLVATATGVHSDVQITQSPSYLAASPGETITINCRAS | 77 |
| VL | KSISKYLAWYQEKPGKTIKLLIYAGSTLQSGIPSRFSGSGYGTDF | |
| | TLTISSLEPEDFAMYYCQQHNEYPLTFGAGTKLELK | |
| 89-E8C12- | MGWSCIILFLVATATGVHSEVQLQQSGPELVKPGASVKISCKASG | 78 |
| VH | YDFTGYYMNWMKQSPEKSLEWIGEINPITSHNTFNQKFKAKATLT | |
| | VDRSSSTAYMHLKSLTSEDSAVYYCARRDYGTSLDYWGQGTTLTV | |
| | SS | |
| 89-E8C12-VL | MGWSCIILFLVATATGVHSDVQITQSPSYLAASPGETITIYCRAS | 79 |
| | KSISKYLAWYQEKPEKTIRLLIYAGSTLQSGIPSRFRGSGSGTDF | |
| | TLTVSGLEPEDFAMYYCQQHNEYPLTFGAGTKLELK | |
| 85-D5A10- | MGWSCIILFLVATATGVHSEVQLQQSGPELVKPGASVKISCKASG | 80 |
| VH | YEFTGYHMNWVKQSPEKSLEWIGEINPITSDKTFNQKFKAKATLT | |
| | VDRSSSTAYMHLKSLTSDDSAVYYCARRDYGTSLDYWGQGTTLTV | |
| | SS | |
| 85-D5A10- | MGWSCIILFLVATATGVHSDVQITQSPSYLAASPGETITIYCRAS | 81 |
| VL | KSISKYLAWYQEKPEKTIRLLIYAGSTLQSGIPSRFRGSGSGKDF | |
| | TLTVSGLEPEDFAMYYCQQHNEYPITFGAGTKLELI | |
| 66-D1B10- | MGWSCIILFLVATATGVHSEVRLEESGGGFVQPGGSMKLSCVASG | 82 |
| VH | LTFPNHWMNWVRQSPERGLAWVAQIQLKSDNYATRYVESVKGRFT | |
| | ISRDDSESTVYLQMNNLRAEDTGIYYCTTGTDYWGQGTTLTVSS | |
| 66-D1B10- | MGWSCIILFLVATATGVHSDVVMTQTPLTLSLTIGQPASISCKSS | 83 |
| VL | QSLLYSDGKTYLNWLLQRPGQSPKRLIYLVSNMDSGVPDRFTGSG | |
| | SGTDFTLKISRVEAEDLGIYYCVQGTHFPYTFGGGTKLEIK | |
| 63-B10B9- | MGWSCIILFLVATATGVHSEVQLQQSGPELVKPGASVKISCKASG | 84 |
| VH | YSFTGYYMNWVKQSPEKSLEWIGEINPITGGTTYNQKFRAKATLT | |
| | VDRSSSTAYMQLKSLTSEDSAVYYCTKRDYDTSLDYWGQGTTLIV | |
| | SS | |
| 63-B10B9-VL | MGWSCIILFLVATATGVHSDVQITQSPSYLAAFPGETITINCRAS | 85 |
| | KSIKKYLAWYQEKPGKTNNLLIYSGSTLQSGIPSRFSGSGTGTDF | |
| | TLTISSLEPEDFAMYYCQQHNEYPLTFGAGTKLELK | |
| 70-F4E5-VH | MGWSCIILFLVATATGVHSEVNLEESGGGLVQPGGSRQLSCVASG | 66 |
| | LTFSKHWMNWVRQSPDKGLEWVAQIQLKSDNYAIRYVDSVKGRFT | |
| | ISRDDSENTVYLQMNNLRAEDTGIYYCTTGTDYWGQGTTLTVSS | |
| 70-F4E5-VL | MGWSCIILFLVATATGVHSDVVMTQTPLTLSVTIGQPASISCRSS | 86 |
| | QSLLYSNGKTYLNWLLQRPGQSPKRLIYLVSNLDSGVPDRFIGSG | |
| | SGTDFTLKISRVEAEDLGVYYCVQGTHFPYTFGGGTKLEIK | |
| 65-F3C7-VH | MGWSCIILFLVATATGVHSEVRLEESGGGFVQPGGSMKLSCVASG | 87 |
| | LTFPNHWMNWVRQSLERGLAWVAQIQLKSDNYATRYVESVKGRFT | |
| | ISRDDSKSTVYLQMNNLRPEDTGIYYCTTGTDYWGQGTTLTVSS | |
| 65-F3C7-VL | MGWSCIILFLVATATGVHSDVVMTQTPLTLSLTIGQPASISCKSS | 83 |
| | QSLLYSDGKTYLNWLLQRPGQSPKRLIYLVSNMDSGVPDRFTGSG | |
| | SGTDFTLKISRVEAEDLGIYYCVQGTHFPYTFGGGTKLEIK | |
| 67-H2E7-VH | MGWSCIILFLVATATGVHSEVQLQQSGPELVKPGASVKISCKASG | 88 |
| | YEFTGYHMNWVKQSPEKSLEWIGEINPITSDKTFNQKFKAKATLT | |
| | VDRSSTTAYMHLKSLTSEDSAVYYCARRDYDTSLDYWGQGTTLTV | |
| | SS | |
| 67-H2E7-VL | MGWSCIILFLVATATGVHSDVQITQSPSYLAASPGETITIYCRAS | 89 |
| | KSISKYLAWYQEKPEKTNRLLIYAGSTLQSGIPSRFRGSGSGKDF | |
| | TLTVSGLEPEDFAMYYCQQHNEYPLTFGAGTKLDLK | |
| 63-C5A5-VH | MGWSCIILFLVATATGVHSEVNLEESGGGLVQPGGSMEFSCVASG | 90 |
| | LTFSDHWMNWVRQSPEKGLEWVAQIQLKSDNYAIRYVESVKGRES | |
| | ISRDDSKSSVYLQMNNLRAEDSGIYYCTTGTDYWGQGTTLTVSS | |
| 63-C5A5-VL | MGWSCIILFLVATATGVHSDVVMTQTPLTLSVTIGQPASISCKSS | 91 |
| | QSLLYSNGKTYLNWLLQRPGQSPKRLIYLVSKLDSGVPDRFTGSG | |
| | SGTDFTLKISRVEAEDLGVYYCIQGTHFPYTFGGGTKLEIK | |
| 69-D5G10- | MGWSCIILFLVATATGVHSEVKLEESGGGLVQPGGSMKLSCVVSG | 92 |
| VH | LTFSNHWMNWVRQSPEKGLEWVAQIQLKSDNYATRFAESVKGRFT | |
| | ISRDDSKSDVYLQMNNLRTEDTGIYYCTTGTDYWGQGTTLTVSS | |
| 69-D5G10- | MGWSCIILFLVATATGVHSDVVMTQTPLTLSVTIGQPASISCKSS | 93 |
| VL | QSLLYSNGKTYLNWLLQRPGQSPKRLIYLVSNLDSGVPDRFTGSG | |
| | SGTDFTLKISRVEAEDLGVYYCIQGTHEPYTFGGGTKLEIK | |

### Example 2. Binding Properties of Anti-CD24 Mouse Monoclonal Antibodies

This example tested the binding properties of the mouse anti-CD24 antibodies to the human CD24 proteins via tumor cell phagocytosis by macrophages, in which CD24⁺ tumor cells (HEK-293, NALM6, OVCAR3 and MCF-7) were co-cultured with human differentiated macrophage in the presence or absence of anti-CD24 mAbs fused with human IgG1 or IgG4. The results are summarized in Table 3, which shows EC₅₀ of the phagocytosis assay. Both NS-17(SWA11) and pp 6373 are anti-CD24 mAbs.

TABLE 3
Tumor cell phagocytosis by macrophages

| | EC50 (μg/mL) of Phagocytosis assay, IgG1, Kappa | | | |
|---|---|---|---|---|
| Clone number | HEK293-CD24 | OVCAR3 | NALM6 | MCF-7 |
| NS-17(SWA11) | | 0.91 | 1.01 | 0.02 |
| PP6373 | | 0.27 | 0.02 | 0 |
| 80-F10A6 | 0.39 | 2.23 | 0.46 | 0.57 |
| 89-E8C12 (4428) | 0.2 | 0.15 | 0.23 | 0.33 |
| 74-H4C6 (4451) | 0.05 | 0.1 | 0.19 | 0.28 |
| 76-F8C6 | 0.09 | 0.17 | 0.42 | 0.47 |
| 75-H8B9 | 0.08 | 0.14 | 1.16 | 0.44 |
| 79-E3H3 | 0.19 | 0.44 | 0.49 | 0.52 |
| 90-D11B6 | 1.31 | 0.59 | 1.19 | 1 |
| 85-D5A10 (4434) | 6.76 | 0.13 | 0.32 | 0.42 |
| 59-G9D7 | 4.76 | 0.97 | 7.9 | 2.05 |
| 62-F7E9 | 5.15 | 1.63 | 8.62 | 2.08 |
| 45-H1E4 | 0.29 | 0.59 | 1.25 | 1.02 |
| 66-D1B10 | 7.46 | 0.73 | 2.48 | 0.84 |
| 63-B10B9 | 7.02 | 0.56 | 1.91 | 1.06 |
| 70-F4E5 | 0.04 | 0.15 | 0.61 | 0.7 |
| 65-F3C7 | 0.75 | 0.23 | 2.01 | 0.4 |
| 67-H2E7 | 0.42 | 8.46 | 0.22 | 0.45 |
| 63-C5A5 | 0.4 | 8.48 | 0.7 | 0.74 |
| 69-D5G10 | 0.22 | 8.44 | 0.43 | 0.42 |
| | EC50 (μg/mL) of Phagocytosis assay, IgG4, Kappa | | | |
| | HEK293-CD24 | OVCAR3 | NALM6 | MCF-7 |
| NS-17(SWA11) | | 0 | 0.18 | 0.09 |
| PP6373 | | 0 | 0.08 | 0 |
| 80-F10A6 | 0.86 | 0.37 | 1.05 | 2.4 |
| 89-E8C12 | 0.43 | 0.16 | 0.57 | 0.96 |
| 74-H4C6 | 0.44 | 0.16 | 0.32 | 0.74 |
| 76-F8C6 | 0.4 | 0.27 | 1.62 | 2.44 |
| 75-H8B9 | 0.4 | 0.24 | 0.84 | 0.76 |
| 79-E3H3 | 0.58 | 0.42 | 0.72 | 1.42 |
| 90-D11B6 | 2.78 | 0.86 | 1.31 | 1.94 |
| 85-D5A10 | 0.42 | 0.38 | 0.47 | 0.43 |
| 59-G9D7 | 2.08 | 1.26 | 6.57 | 0.19 |
| 62-F7E9 | 10.52 | 1.41 | 0.01 | 0.21 |
| 45-H1E4 | 0.9 | 3.35 | 5.36 | 7.00 |
| 66-D1B10 | 3.09 | 5.77 | 6.28 | 2.26 |
| 63-B10B9 | 1.14 | 9.85 | 6.16 | 17.07 |
| 70-F4E5 | 0.21 | 5.61 | 0.63 | 0.84 |
| 65-F3C7 | 0.09 | 5.74 | 6.01 | 2.25 |
| 67-H2E7 | 0.51 | 0.71 | 0.68 | 1.28 |
| 63-C5A5 | 0.46 | 1.62 | 6.58 | 24.67 |
| 69-D5G10 | 0.37 | 0.51 | 0.55 | 1.02 |

The results of tumor cells binding assay are summarized in Table 4.

TABLE 4
Binding on the tumor cells.

| | MCF-7 binding | | NALM6 cell binding | | B cell binding |
|---|---|---|---|---|---|
| Clone number | IgG1, Kappa | IgG4, Kappa | IgG1, Kappa | IgG4, Kappa | IgG1, Kappa |
| 80-F10A6 | 0.41 | 1.17 | 0.41 | 0.45 | 0.15 |
| 89-E8C12 (4428) | 0.31 | 0.31 | 0.40 | 0.56 | 0.07 |
| 74-H4C6 (4451) | 0.18 | 0.22 | 0.40 | 0.42 | 0.08 |
| 76-F8C6 | 0.18 | 0.33 | 0.42 | 1.18 | 0.14 |
| 75-H8B9 | 0.30 | 0.35 | 0.41 | 0.62 | 0.18 |
| 79-E3H3 | 0.16 | 0.23 | 0.42 | 0.41 | 0.08 |
| 90-D11B6 | 0.30 | 0.63 | 0.42 | 0.37 | 1.43 |
| 85-D5A10 (4434) | 0.33 | 0.40 | 0.41 | 0.42 | 0.08 |
| 59-G9D7 | 0.39 | 1.62 | 0.83 | 2.98 | 2.47 |
| 62-F7E9 | 0.86 | 0.97 | 0.63 | 2.69 | 2.44 |
| 45-H1E4 | 0.25 | 0.37 | 0.41 | 0.88 | 0.25 |
| 66-D1B10 | 0.29 | 0.34 | 0.81 | 1.62 | 1.91 |
| 63-B10B9 | 1.13 | 0.72 | 2.01 | 1.57 | 0.11 |
| 70-F4E5 | 0.41 | 0.42 | 0.42 | 0.44 | 0.16 |
| 65-F3C7 | 0.40 | 0.68 | 0.80 | 2.63 | 1.28 |
| 67-H2E7 | 0.69 | 0.62 | 0.42 | 0.83 | 0.04 |
| 63-C5A5 | 0.39 | / | 0.59 | 1.07 | 0.45 |
| 69-D5G10 | 0.40 | 0.31 | 0.40 | 1.30 | 0.25 |

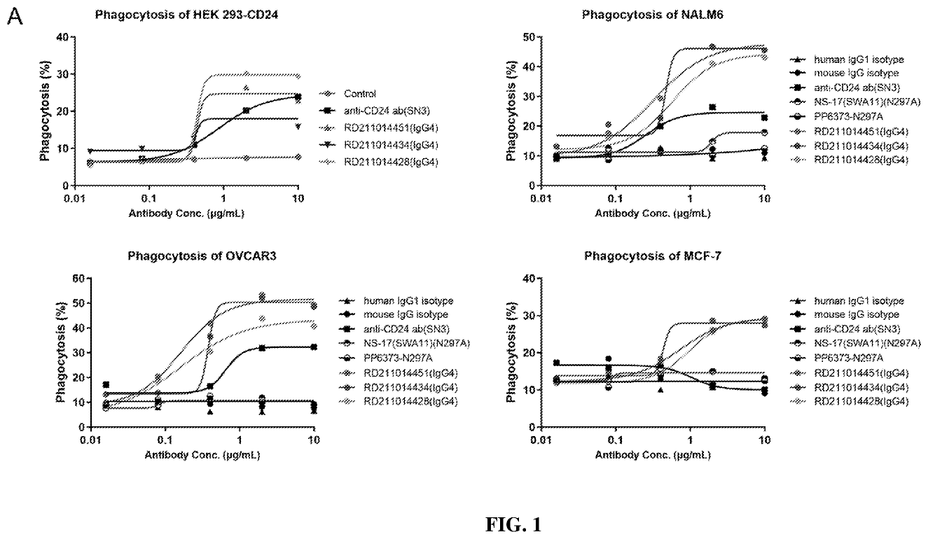

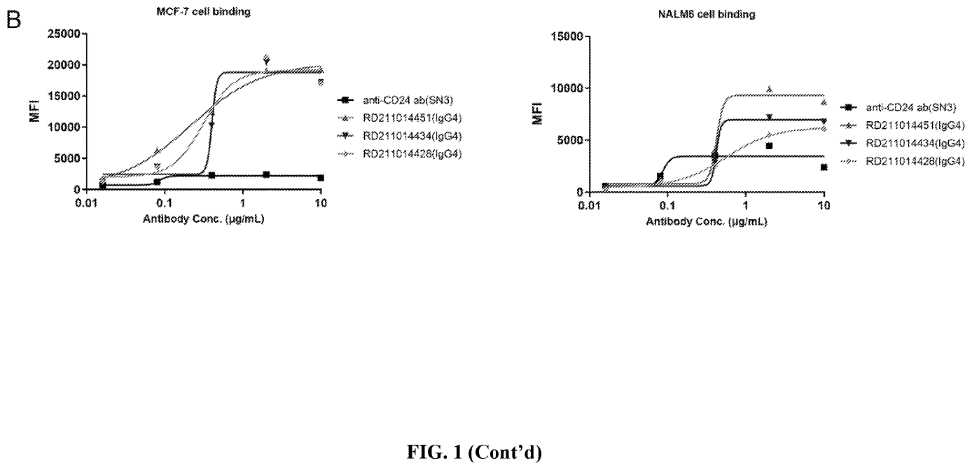

Further phagocytosis (FIG. 1, panel A) and tumor cell binding assays (FIG. 1, panel B) were performed using the selected mAbs fused with human IgG4: 89-E8C12 (4428), 74-H4C6 (4451) and 85-D5A10 (4434), with anti-CD24 antibodies (NS-17 (SWA11), pp 6373 and SN3) and IgG isotypes.

### Example 3. Anti-CD24 mAb Humanization and Affinity Maturation

### Humanization

The mouse antibody 74-H4C6 (4451) variable region genes were employed to create a humanized MAb. In the first step of this process, the amino acid sequences of the V_{H} and VK of 4451 were compared against the available database of human Ig gene sequences to identify the overall best-matching human germline Ig gene sequences.

Humanized variable domain sequences were then designed where the CDR1: GYHMN (SEQ ID NO: 1), CDR2: EINPITSDKTFNQKFKS (SEQ ID NO: 2), CDR3: RDYGTSLDY (SEQ ID NO: 3) sequences of the 4451 V_{H} were grafted onto framework sequences of the human gene and the CDR1: RASKSISKYLA (SEQ ID NO: 4), CDR2: AGSTLHS (SEQ ID NO: 5), and CDR3: QQHNEYPIT (SEQ ID NO: 6) of the 4451 light chain were grafted onto framework sequences of the human gene (Table 5). A 3D model was then generated to determine if there were any framework positions where replacing the mouse amino acid to the human amino acid could affect binding and/or CDR conformation. In the case of the heavy chain, R38, M48, V68, R72, and T74 (Kabat numbering) in human framework was identified and subjected to back-mutations to their mouse counterpart amino acid i.e.: R38K, M48I, V68A, R72V, and T74R. In the case of the light chain, K45, V58, and S63 (Kabat numbering) in human framework was identified and subjected to back-mutation to their moue counterpart amino acid i.e.: K45R, V58I, and S63R.

TABLE 5
4451 sequences and CDRs

| Variable | | SEQ |
|---|---|---|
| region | Sequences* (CDR underlined) | ID NO: |
| 4451-VH | EVQLQQSGPELVKPGASVKISCKASGYDFTGYHMNWVKQSPEKSLEWIGEINPITSD | 60 |
| | KTFNQKFKSKATLTVDRSSSTAYMYLKSLTSEDSAVYYCARRDYGTSLDYWGQGTTL | |
| | TVSS | |
| 4451-VL | DVQITQSPSYLAASPGETITIYCRASKSISKYLAWYQEKPEKTNRLLIYAGSTLHSG | 61 |
| | IPSRFRGSGSGKDFTLTVSGLEPEDFAMYYCQQHNEYPITFGAGTKLELK | |
| CDRH1 | GYHMN | 1 |
| CDRH2 | EINPITSDKTFNQKFKS | 2 |
| CDRH3 | RDYGTSLDY | 3 |
| CDRL1 | RASKSISKYLA | 4 |
| CDRL2 | AGSTLHS | 5 |
| CDRL3 | QQHNEYPIT | 6 |

The humanized sequences are listed in Table 6:4451-VH1, 4451-VH2, 4451-VH3, 4451-VH4, 4451-VL1, 4451-VL2, 4451-VL3, and 4451-VL4.

TABLE 6
Humanized sequences

| Variable | | SEQ |
|---|---|---|
| region | Sequences* (CDR underlined) | ID NO: |
| 4451-VH1 | QVQLVQSGAEVKKPGASVKVSCKASGYDFTGYHMNWVRQAPGQGLEWMGEINPITSD | 94 |
| | KTFNQKFKSRVTMTRDTSISTAYMELSRLRSDDTAVYYCARRDYGTSLDYWGQGTTV | |
| | TVSS | |
| 4451-VH2 | QVQLVQSGAEVKKPGASVKVSCKASGYDFTGYHMNWVRQAPGQGLEWIGEINPITSD | 95 |
| | KTFNQKFKSRVTMTVDTSISTAYMELSRLRSDDTAVYYCARRDYGTSLDYWGQGTTV | |
| | TVSS | |
| 4451-VH3 | QVQLVQSGAEVKKPGASVKVSCKASGYDFTGYHMNWVRQAPGQGLEWIGEINPITSD | 96 |
| | KTFNQKFKSRATMTVDTSISTAYMELSRLRSDDTAVYYCARRDYGTSLDYWGQGTTV | |
| | TVSS | |
| 4451-VH4 | QVQLVQSGAEVKKPGASVKVSCKASGYDFTGYHMNWVKQAPGQGLEWIGEINPITSD | 97 |
| | KTFNQKFKSRATMTVDRSISTAYMELSRLRSDDTAVYYCARRDYGTSLDYWGQGTTV | |
| | TVSS | |
| 4451-VL1 | DIQLTQSPSFLSASVGDRVTITCRASKSISKYLAWYQQKPGKAPKLLIYAGSTLHSG | 98 |
| | VPSRFSGSGSGTEFTLTISSLQPEDFATYYCQQHNEYPITFGQGTKLEIK | |
| 4451-VL2 | DIQLTQSPSFLSASVGDRVTITCRASKSISKYLAWYQQKPGKAPRLLIYAGSTLHSG | 99 |
| | VPSRFRGSGSGTEFTLTISSLQPEDFATYYCQQHNEYPITFGQGTKLEIK | |
| 4451-VL3 | DIQLTQSPSFLSASVGDRVTITCRASKSISKYLAWYQQKPGKAPKLLIYAGSTLHSG | 100 |
| | IPSRFRGSGSGTEFTLTISSLQPEDFATYYCQQHNEYPITFGQGTKLEIK | |
| 4451-VL4 | DIQLTQSPSFLSASVGDRVTIYCRASKSISKYLAWYQQKPGKAPRLLIYAGSTLHSG | 101 |
| | IPSRFRGSGSGTEFTLTISSLQPEDFATYYCQQHNEYPITFGQGTKLEIK | |

All the 16 IgGs were expressed in the HEK293 cell line. Express antibodies were performed for affinity ranking. Detailed data of affinity ranking were summarized in Table 7.

TABLE 7
Affinity ranking of humanized antibodies.

| Ligand | Analyte | ka (1/Ms) | kd (1/s) | KD (M) |
|---|---|---|---|---|
| VH + VL | Human CD24 | 2.90E+06 | 9.37E−03 | 3.23E−09 |
| VH1 + VL1 | Human CD24 | 9.58E+05 | 1.69E−02 | 1.76E−08 |
| VH1 + VL2 | Human CD24 | 1.74E+06 | 2.39E−02 | 1.38E−08 |
| VH1 + VL3 | Human CD24 | 6.85E+05 | 1.58E−02 | 2.31E−08 |
| VH1 + VL4 | Human CD24 | 1.61E+06 | 2.41E−02 | 1.50E−08 |
| VH2 + VL1 | Human CD24 | 1.26E+06 | 2.64E−02 | 2.09E−08 |
| VH2 + VL2 | Human CD24 | 1.06E+06 | 2.34E−02 | 2.20E−08 |
| VH2 + VL3 | Human CD24 | 1.18E+06 | 2.30E−02 | 1.94E−08 |
| VH2 + VL4 | Human CD24 | 1.16E+06 | 2.29E−02 | 1.97E−08 |
| VH3 + VL1 | Human CD24 | 1.13E+06 | 2.94E−02 | 2.60E−08 |
| VH3 + VL2 | Human CD24 | 1.10E+06 | 2.62E−02 | 2.37E−08 |
| VH3 + VL3 | Human CD24 | 1.13E+06 | 2.70E−02 | 2.38E−08 |
| VH3 + VL4 | Human CD24 | 6.55E+05 | 1.63E−02 | 2.48E−08 |
| VH4 + VL1 | Human CD24 | 1.54E+06 | 1.28E−02 | 8.33E−09 |
| VH4 + VL2 | Human CD24 | 1.28E+06 | 1.15E−02 | 9.00E−09 |
| VH4 + VL3 | Human CD24 | 1.34E+06 | 1.19E−02 | 8.84E−09 |
| VH4 + VL4 | Human CD24 | 1.53E+06 | 1.22E−02 | 7.94E−09 |
| NC | Human CD24 | NA | NA | NA |

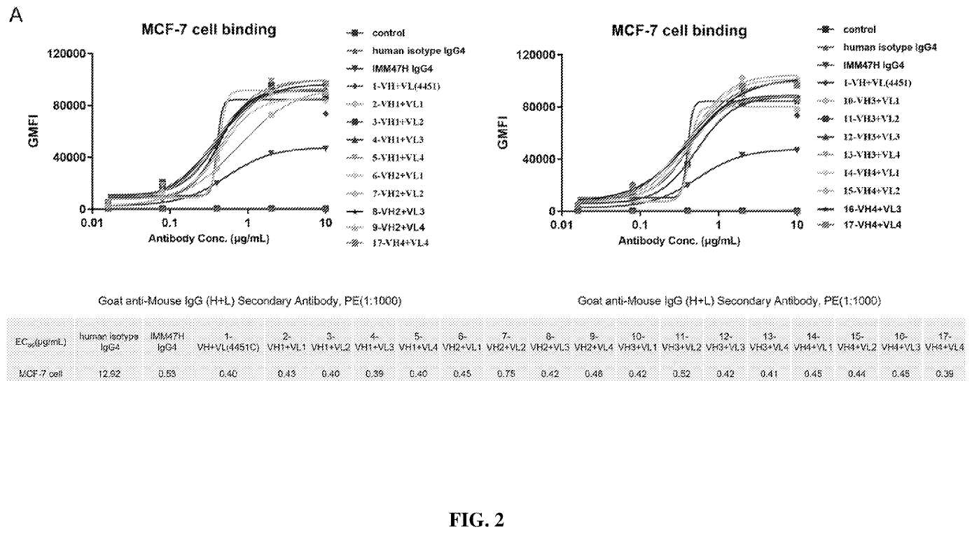

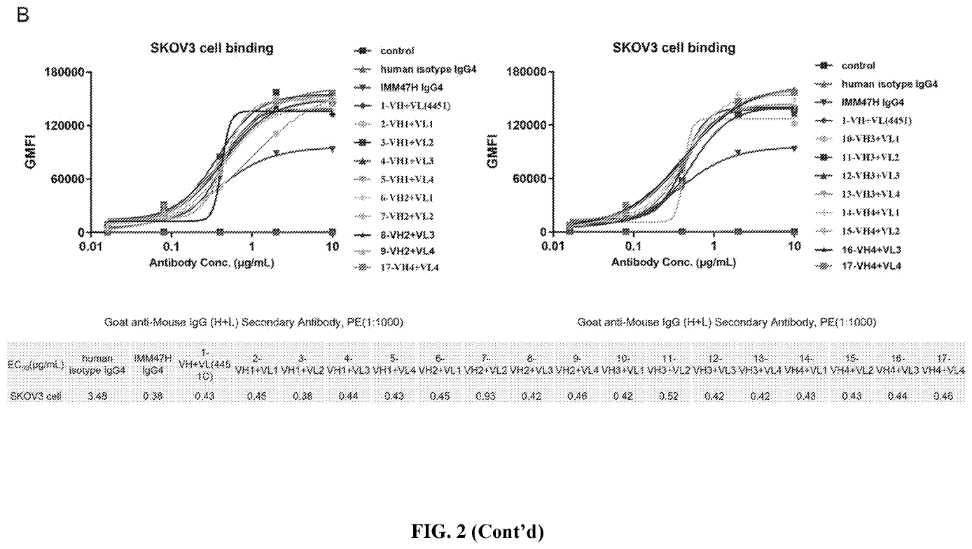

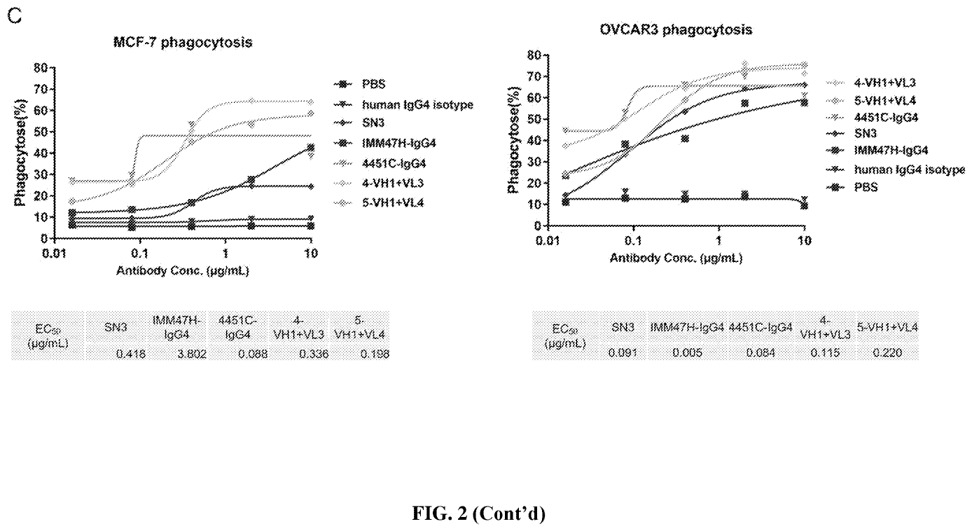

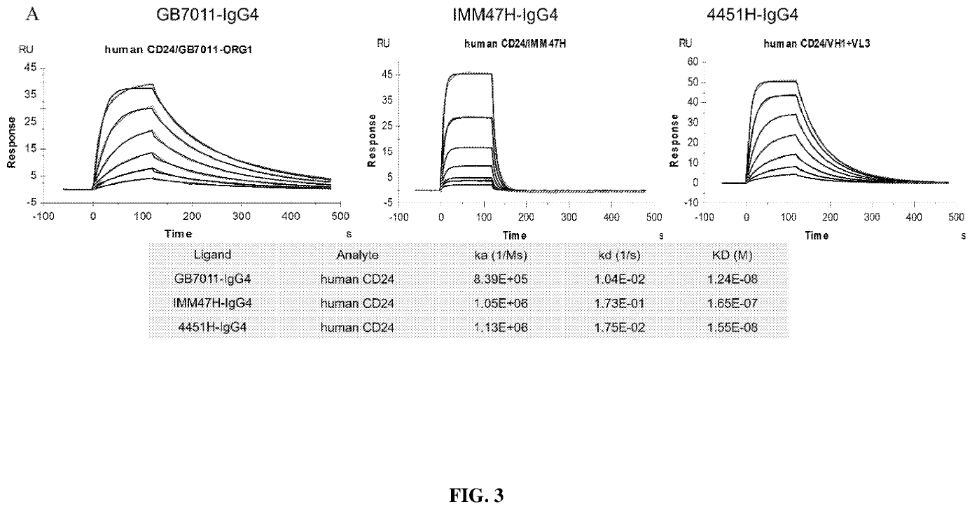

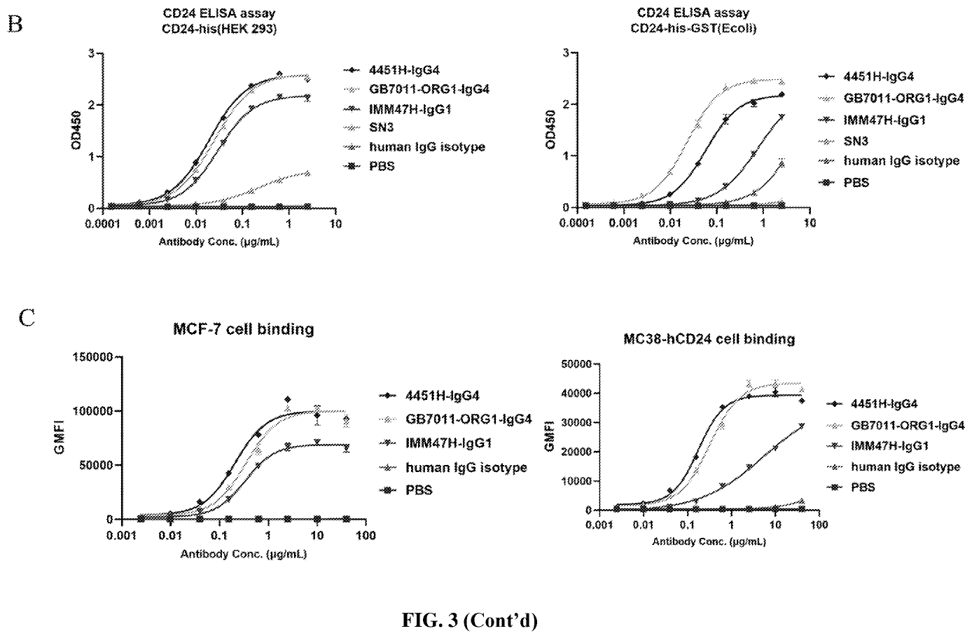

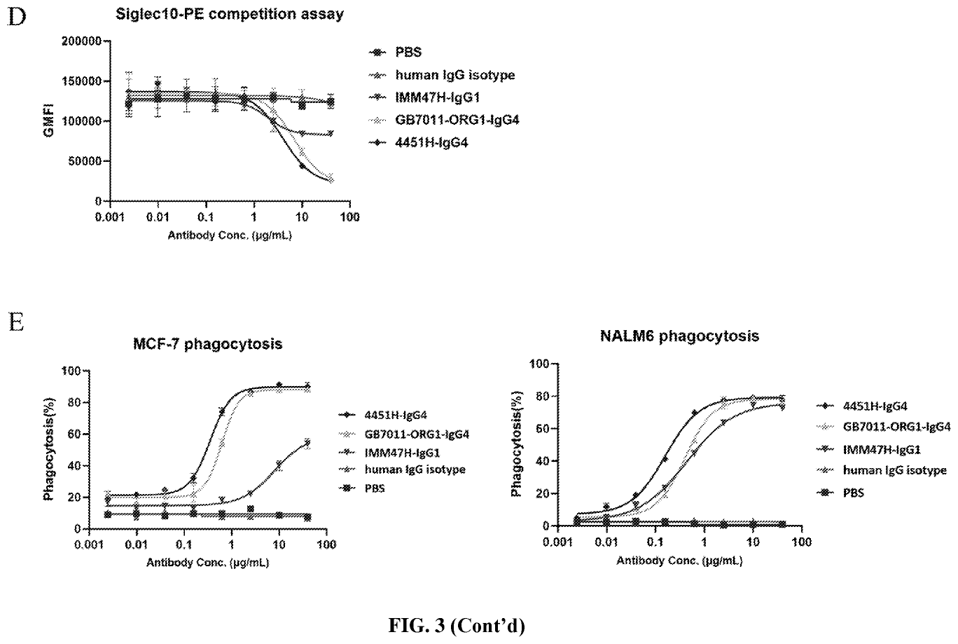

Further tumor cell binding affinity (see FIG. 2, panel A-B) and phagocytosis (see FIG. 2, panel C) of the 16 humanized IgGs were performed. Based on the affinity and phagocytosis results, one IgG (VH1+VL3) were expressed and purified as 4451H, which was fused with human IgG4 Fc and compared with two reference anti-CD24 antibodies (IMM47H, disclosed in WO2023061064A1 and GB7011 (Genbase)) in affinity binding by Biacore (see FIG. 3, panel A-B), tumor cell binding (see FIG. 3, panel C), blocking (see FIG. 3, panel D) and phagocytosis (see FIG. 3, panel E). As shown in the results, 4451H showed better efficacy than the two reference anti-CD24 antibodies in the FACS affinity assay, CD24-Siglec-10 blocking assay and phagocytocis assays.

Efficacy of Anti-CD24 mAb Treatment in Humanized Siglec-10 Mice Bearing hCD24 Overexpressed MC38 Tumor Model (Batch 1 #)

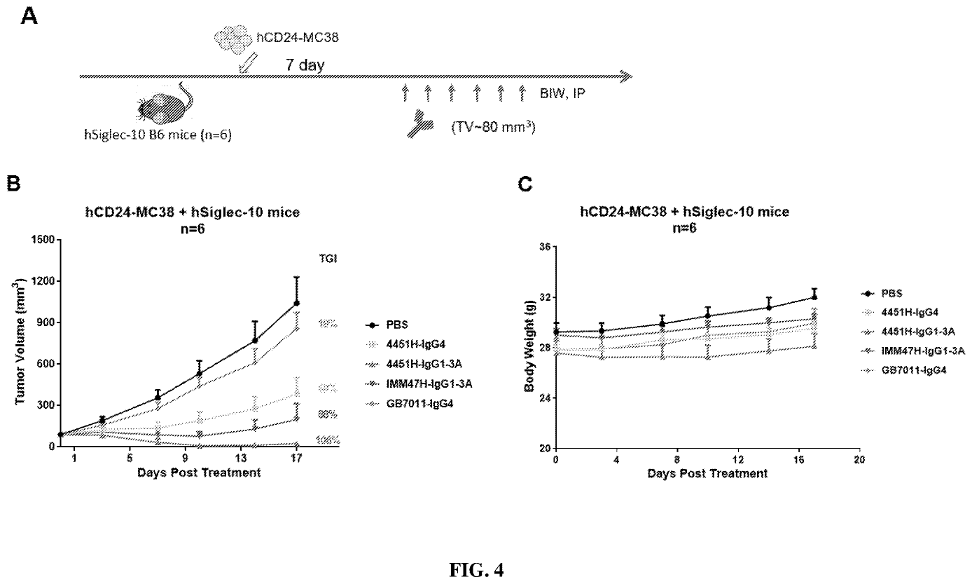

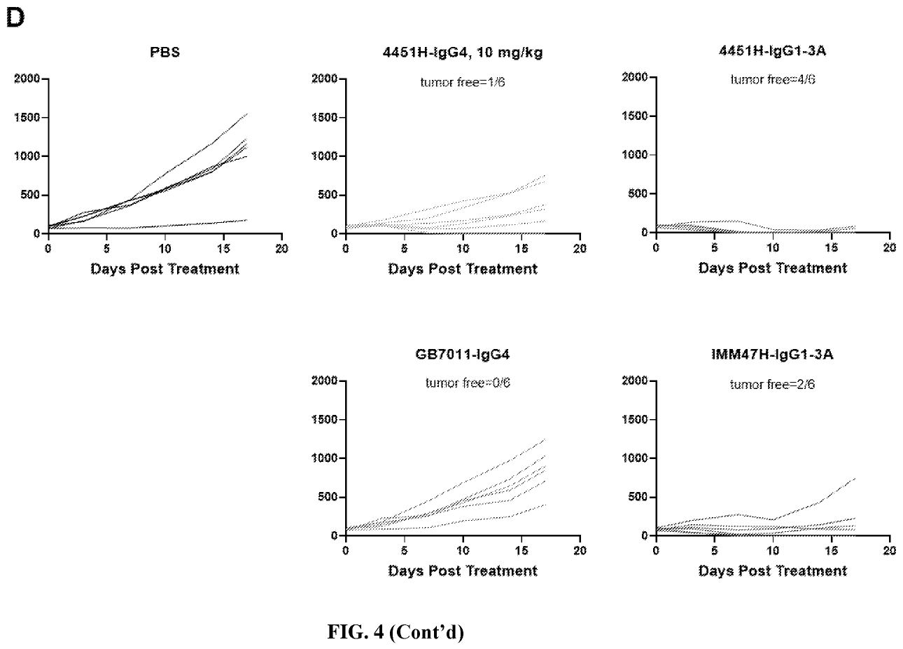

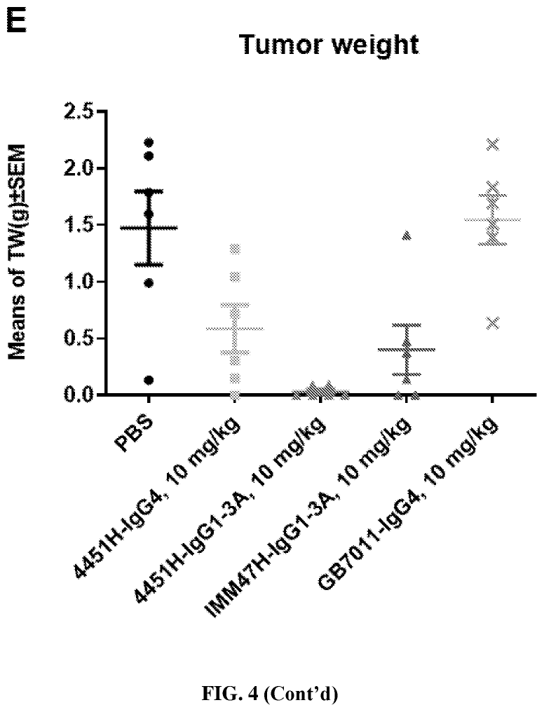

To determine whether the in vitro effects of anti-CD24 mAb translated into in vivo anti-tumor activity, a Siglec-10 humanized C57BL/6 mice bearing human CD24 protein overexpressed MC38 tumor cells was employed. Briefly, hSiglec-10 mice were subcutaneously engrafted with hCD24-MC38 cells. When the tumor volume reached about 80 mm3, anti-CD24 antibodies or PBS were intraperitoneally administrated twice a week for 3 weeks. The tumor volume was monitored twice per week during the experiment. In this experiment, humanized 4451 sequence (4451H) was constructed to human IgG4 and human IgG1 (S298A/E333A/K334A) to evaluate the efficacy. And two other antibodies, GB7011-IgG4 and IMM47H-IgG1-3A were also tested parallelly. As shown in FIG. 4 (panel A-E), both 4451H-IgG4 and 4451H-IgG1-3A treatment markedly reduced tumor growth compared with control group, achieving 69% and 106% tumor growth inhibition (TGI), respectively. What's more, two mice from 4451H-IgG4 group and four mice from 4451H-IgG1-3A group achieved tumor free treatment.

Taken together, 4451H-IgG4 demonstrates excellent in vivo efficacy, underscoring the importance of CD24/Siglec-10 signal in tumor development; 4451H-IgG1-3A is more potent than 4451H-IgG4.

hSiglec-10 C57 mice + hCD24-MC38

| | Treatment | TGI % | Tumor free |
|---|---|---|---|
| 1 | PBS | — | 0/6 |
| 2 | 4451H-IgG4 | 69 | 1/6 |
| 3 | 4451H-IgG1-3A | 106 | 4/6 |
| 4 | IMM47H-IgG1-3A | 88 | 2/6 |
| 5 | GB7011-IgG4 | 19 | 0/6 |

### Affinity Maturation of 4451H

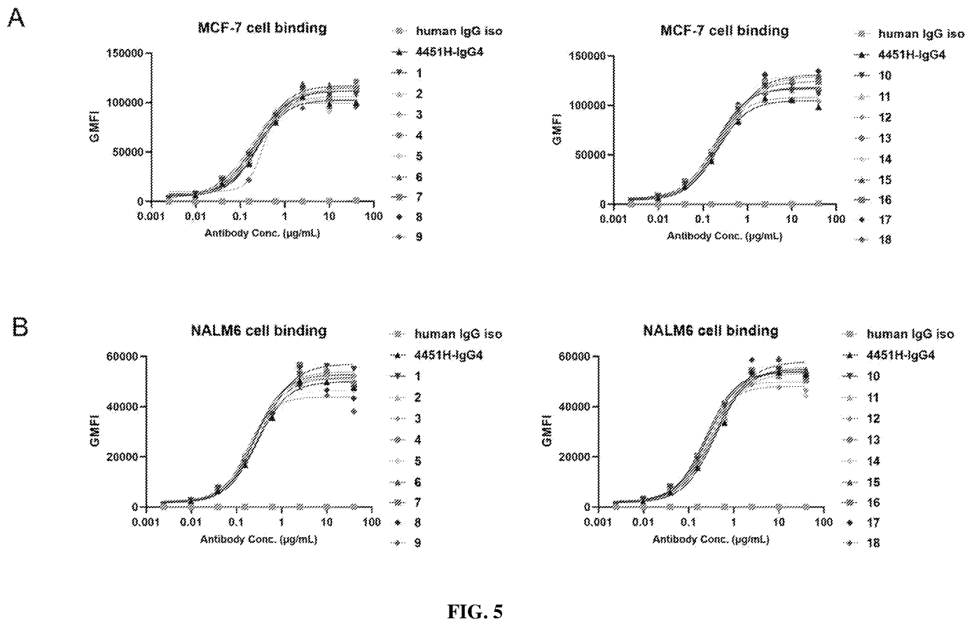

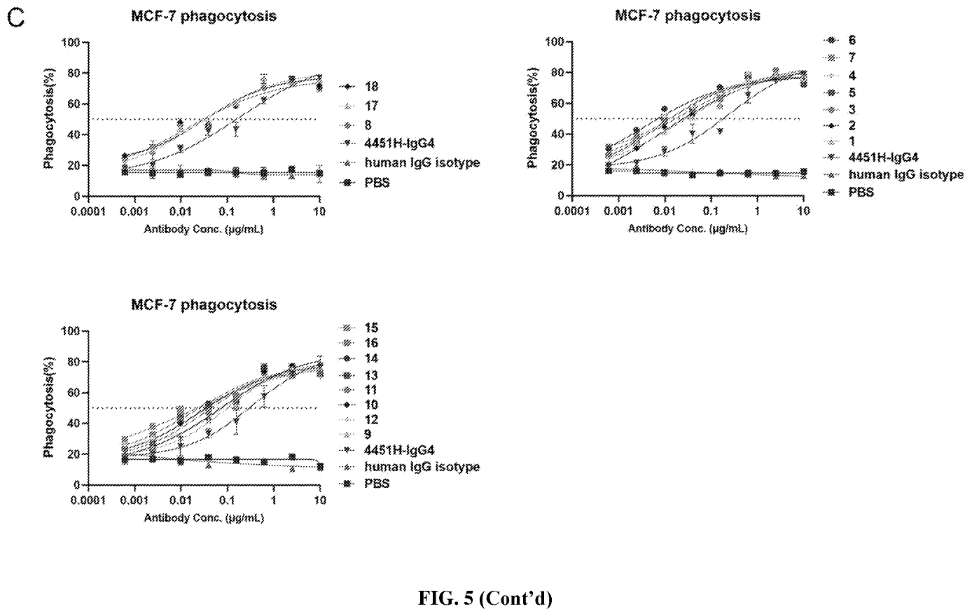

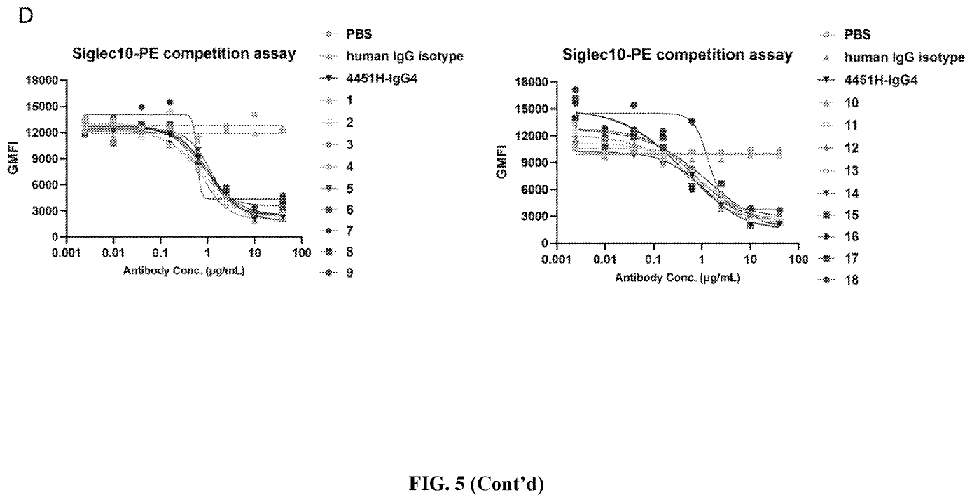

To optimize the K_{off} of 4451H, this example initiated an affinity maturation procedure. Briefly, paratope mapping by using alanine scanning in the CDR region was performed to identify the key residues that affect antibodies binding to CD24 or production. Then the CDR amino acids surrounding the key residues were selected to construct NNK library and screened by affinity ranking to identify mutations that improve the off-rate for human CD24 but do not affect the expression level of the antibody. Mutated Amino acids that could improve K_{off} binding of 4451H are listed in Table 8. A combinational library that incorporated all the mutant form of these amino acids was constructed and screened. Sequences of lead clones that have lower off-rate for human CD24 are listed in Table 9. Antibodies of these sequences were generated and affinity ranking was performed by SPR analysis. The results are listed in Table 10. As described here, antibody No. 15 shows enhanced K_{off}-rate compared with parental antibodies and was chosen and named as 4451HM15. Further tumor cells binding affinity (see FIG. 5, panel A-B) and phagocytosis (see FIG. 5, panel C) and CD24 and siglec10 block assay (see FIG. 5, panel D) of the 18 affinity matured antibodies were performed.

TABLE 8
Mutations in 4451H useful for improving binding

| Residue | Substituted with |
|---|---|
| VH-35N | G |
| VH-59T | Y |
| VL-27K | A |
| VL-30S | K |
| VL-33L | G |
| VL-97T | I |

TABLE 9
Lead clones of 4451H

| Antibody | Antibody | | SEQ ID |
|---|---|---|---|
| No | chain | Sequence of variable region | NO: |
| 0 | VH | QVQLVQSGAEVKKPGASVKVSCKASGYDFTGYHMNWVRQAPGQGLEWMGEI | 94 |
| (4451H) | (4451-VH1) | NPITSDKTFNQKFKSRVTMTRDTSISTAYMELSRLRSDDTAVYYCARRDYG | |
| | | TSLDYWGQGTTVTVSS | |
| | VL | DIQLTQSPSFLSASVGDRVTITCRASKSISKYLAWYQQKPGKAPKLLIYAG | 100 |
| | (4451-VL3) | STLHSGIPSRFRGSGSGTEFTLTISSLQPEDFATYYCQQHNEYPITFGQGT | |
| | | KLEIK | |
| 1 | VH | QVQLVQSGAEVKKPGASVKVSCKASGYDFTGYHMGWVRQAPGQGLEWMGEI | 102 |
| | | NPITSDKTFNQKFKSRVTMTRDTSISTAYMELSRLRSDDTAVYYCARRDYG | |
| | | TSLDYWGQGTTVTVSS | |
| | VL | DIQLTQSPSFLSASVGDRVTITCRASASISKYLAWYQQKPGKAPKLLIYAG | 103 |
| | | STLHSGIPSRFRGSGSGTEFTLTISSLQPEDFATYYCQQHNEYPIIFGQGT | |
| | | KLEIK | |
| 2 | VH | QVQLVQSGAEVKKPGASVKVSCKASGYDFTGYHMGWVRQAPGQGLEWMGEI | 102 |
| | | NPITSDKTFNQKFKSRVTMTRDTSISTAYMELSRLRSDDTAVYYCARRDYG | |
| | | TSLDYWGQGTTVTVSS | |
| | VL | DIQLTQSPSFLSASVGDRVTITCRASKSIKKYLAWYQQKPGKAPKLLIYAG | 104 |
| | | STLHSGIPSRFRGSGSGTEFTLTISSLQPEDFATYYCQQHNEYPIIFGQGT | |
| | | KLEIK | |
| 3 | VH | QVQLVQSGAEVKKPGASVKVSCKASGYDFTGYHMGWVRQAPGQGLEWMGEI | 102 |
| | | NPITSDKTFNQKFKSRVTMTRDTSISTAYMELSRLRSDDTAVYYCARRDYG | |
| | | TSLDYWGQGTTVTVSS | |
| | VL | DIQLTQSPSFLSASVGDRVTITCRASKSISKYGAWYQQKPGKAPKLLIYAG | 105 |
| | | STLHSGIPSRFRGSGSGTEFTLTISSLQPEDFATYYCQQHNEYPIIFGQGT | |
| | | KLEIK | |
| 4 | VH | QVQLVQSGAEVKKPGASVKVSCKASGYDFTGYHMGWVRQAPGQGLEWMGEI | 102 |
| | | NPITSDKTFNQKFKSRVTMTRDTSISTAYMELSRLRSDDTAVYYCARRDYG | |
| | | TSLDYWGQGTTVTVSS | |
| | VL | DIQLTQSPSFLSASVGDRVTITCRASASISKYLAWYQQKPGKAPKLLIYAG | 106 |
| | | STLHSGIPSRFRGSGSGTEFTLTISSLQPEDFATYYCQQHNEYPITFGQGT | |
| | | KLEIK | |
| 5 | VH | QVQLVQSGAEVKKPGASVKVSCKASGYDFTGYHMGWVRQAPGQGLEWMGEI | 102 |
| | | NPITSDKTFNQKFKSRVTMTRDTSISTAYMELSRLRSDDTAVYYCARRDYG | |
| | | TSLDYWGQGTTVTVSS | |
| | VL | DIQLTQSPSFLSASVGDRVTITCRASKSIKKYLAWYQQKPGKAPKLLIYAG | 107 |
| | | STLHSGIPSRFRGSGSGTEFTLTISSLQPEDFATYYCQQHNEYPITFGQGT | |
| | | KLEIK | |
| 6 | VH | QVQLVQSGAEVKKPGASVKVSCKASGYDFTGYHMGWVRQAPGQGLEWMGEI | 102 |
| | | NPITSDKTENQKFKSRVTMTRDTSISTAYMELSRLRSDDTAVYYCARRDYG | |
| | | TSLDYWGQGTTVTVSS | |
| | VL | DIQLTQSPSFLSASVGDRVTITCRASKSISKYGAWYQQKPGKAPKLLIYAG | 108 |
| | | STLHSGIPSRFRGSGSGTEFTLTISSLQPEDFATYYCQQHNEYPITFGQGT | |
| | | KLEIK | |
| 7 | VH | QVQLVQSGAEVKKPGASVKVSCKASGYDFTGYHMGWVRQAPGQGLEWMGEI | 102 |
| | | NPITSDKTENQKFKSRVTMTRDTSISTAYMELSRLRSDDTAVYYCARRDYG | |
| | | TSLDYWGQGTTVTVSS | |
| | VL | DIQLTQSPSFLSASVGDRVTITCRASKSISKYLAWYQQKPGKAPKLLIYAG | 109 |
| | | STLHSGIPSRFRGSGSGTEFTLTISSLQPEDFATYYCQQHNEYPIIFGQGT | |
| | | KLEIK | |
| 8 | VH | QVQLVQSGAEVKKPGASVKVSCKASGYDFTGYHMNWVRQAPGQGLEWMGEI | 110 |
| | | NPITSDKYFNQKFKSRVTMTRDTSISTAYMELSRLRSDDTAVYYCARRDYG | |
| | | TSLDYWGQGTTVTVSS | |
| | VL | DIQLTQSPSFLSASVGDRVTITCRASASISKYLAWYQQKPGKAPKLLIYAG | 103 |
| | | STLHSGIPSRFRGSGSGTEFTLTISSLQPEDFATYYCQQHNEYPIIFGQGT | |
| | | KLEIK | |
| 9 | VH | QVQLVQSGAEVKKPGASVKVSCKASGYDFTGYHMNWVRQAPGQGLEWMGEI | 110 |
| | | NPITSDKYFNQKFKSRVTMTRDTSISTAYMELSRLRSDDTAVYYCARRDYG | |
| | | TSLDYWGQGTTVTVSS | |
| | VL | DIQLTQSPSFLSASVGDRVTITCRASKSIKKYLAWYQQKPGKAPKLLIYAG | 104 |
| | | STLHSGIPSRFRGSGSGTEFTLTISSLQPEDFATYYCQQHNEYPIIFGQGT | |
| | | KLEIK | |
| 10 | VH | QVQLVQSGAEVKKPGASVKVSCKASGYDFTGYHMNWVRQAPGQGLEWMGEI | 110 |
| | | NPITSDKYFNQKFKSRVTMTRDTSISTAYMELSRLRSDDTAVYYCARRDYG | |
| | | TSLDYWGQGTTVTVSS | |
| | VL | DIQLTQSPSFLSASVGDRVTITCRASKSISKYGAWYQQKPGKAPKLLIYAG | 105 |
| | | STLHSGIPSRFRGSGSGTEFTLTISSLQPEDFATYYCQQHNEYPIIFGQGT | |
| | | KLEIK | |
| 11 | VH | QVQLVQSGAEVKKPGASVKVSCKASGYDFTGYHMNWVRQAPGQGLEWMGEI | 110 |
| | | NPITSDKYFNQKFKSRVTMTRDTSISTAYMELSRLRSDDTAVYYCARRDYG | |
| | | TSLDYWGQGTTVTVSS | |
| | VL | DIQLTQSPSFLSASVGDRVTITCRASASISKYLAWYQQKPGKAPKLLIYAG | 106 |
| | | STLHSGIPSRFRGSGSGTEFTLTISSLQPEDFATYYCQQHNEYPITFGQGT | |
| | | KLEIK | |
| 12 | VH | QVQLVQSGAEVKKPGASVKVSCKASGYDFTGYHMNWVRQAPGQGLEWMGEI | 110 |
| | | NPITSDKYFNQKFKSRVTMTRDTSISTAYMELSRLRSDDTAVYYCARRDYG | |
| | | TSLDYWGQGTTVTVSS | |
| | VL | DIQLTQSPSFLSASVGDRVTITCRASKSIKKYLAWYQQKPGKAPKLLIYAG | 107 |
| | | STLHSGIPSRFRGSGSGTEFTLTISSLQPEDFATYYCQQHNEYPITFGQGT | |
| | | KLEIK | |
| 13 | VH | QVQLVQSGAEVKKPGASVKVSCKASGYDFTGYHMNWVRQAPGQGLEWMGEI | 110 |
| | | NPITSDKYFNQKFKSRVTMTRDTSISTAYMELSRLRSDDTAVYYCARRDYG | |
| | | TSLDYWGQGTTVTVSS | |
| | VL | DIQLTQSPSFLSASVGDRVTITCRASKSISKYGAWYQQKPGKAPKLLIYAG | 108 |
| | | STLHSGIPSRFRGSGSGTEFTLTISSLQPEDFATYYCQQHNEYPITFGQGT | |
| | | KLEIK | |
| 14 | VH | QVQLVQSGAEVKKPGASVKVSCKASGYDFTGYHMNWVRQAPGQGLEWMGEI | 110 |
| | | NPITSDKYFNQKFKSRVTMTRDTSISTAYMELSRLRSDDTAVYYCARRDYG | |
| | | TSLDYWGQGTTVTVSS | |
| | VL | DIQLTQSPSFLSASVGDRVTITCRASKSISKYLAWYQQKPGKAPKLLIYAG | 109 |
| | | STLHSGIPSRFRGSGSGTEFTLTISSLQPEDFATYYCQQHNEYPIIFGQGT | |
| | | KLEIK | |
| 15 | VH | QVQLVQSGAEVKKPGASVKVSCKASGYDFTGYHMGWVRQAPGQGLEWMGEI | 111 |
| (4451HM15) | | NPITSDKYFNQKFKSRVTMTRDTSISTAYMELSRLRSDDTAVYYCARRDYG | |
| | | TSLDYWGQGTTVTVSS | |
| | VL | DIQLTQSPSFLSASVGDRVTITCRASASISKYLAWYQQKPGKAPKLLIYAG | 106 |
| | | STLHSGIPSRFRGSGSGTEFTLTISSLQPEDFATYYCQQHNEYPITFGQGT | |
| | | KLEIK | |
| 16 | VH | QVQLVQSGAEVKKPGASVKVSCKASGYDFTGYHMGWVRQAPGQGLEWMGEI | 111 |
| | | NPITSDKYFNQKFKSRVTMTRDTSISTAYMELSRLRSDDTAVYYCARRDYG | |
| | | TSLDYWGQGTTVTVSS | |
| | VL | DIQLTQSPSFLSASVGDRVTITCRASKSIKKYLAWYQQKPGKAPKLLIYAG | 107 |
| | | STLHSGIPSRFRGSGSGTEFTLTISSLQPEDFATYYCQQHNEYPITFGQGT | |
| | | KLEIK | |
| 17 | VH | QVQLVQSGAEVKKPGASVKVSCKASGYDFTGYHMGWVRQAPGQGLEWMGEI | 111 |
| | | NPITSDKYFNQKFKSRVTMTRDTSISTAYMELSRLRSDDTAVYYCARRDYG | |
| | | TSLDYWGQGTTVTVSS | |
| | VL | DIQLTQSPSFLSASVGDRVTITCRASKSISKYGAWYQQKPGKAPKLLIYAG | 108 |
| | | STLHSGIPSRFRGSGSGTEFTLTISSLQPEDFATYYCQQHNEYPITFGQGT | |
| | | KLEIK | |
| 18 | VH | QVQLVQSGAEVKKPGASVKVSCKASGYDFTGYHMGWVRQAPGQGLEWMGEI | 111 |
| | | NPITSDKYFNQKFKSRVTMTRDTSISTAYMELSRLRSDDTAVYYCARRDYG | |
| | | TSLDYWGQGTTVTVSS | |
| | VL | DIQLTQSPSFLSASVGDRVTITCRASKSISKYLAWYQQKPGKAPKLLIYAG | 109 |
| | | STLHSGIPSRFRGSGSGTEFTLTISSLQPEDFATYYCQQHNEYPIIFGQGT | |
| | | KLEIK | |

TABLE 10
Binding results

| | | | | | | | 1:1 | | |
|---|---|---|---|---|---|---|---|---|---|
| | | LC | Analyte | 1:1 binding | | | binding | | |
| No. | HC mutation | mutation | Conc. | ka (1/Ms) | kd (1/s) | KD (M) | ka (1/Ms) | kd (1/s) | KD (M) |
| 0 | Parental | Parental | 19.71 nM | 1.60E+05 | 2.75E−02 | 1.72E−07 | 7.63E+05 | 1.92E−02 | 2.51E−08 |
| 1 | N35G | K27A, T97I | 19.71 nM | 1.26E+06 | 1.32E−02 | 1.05E−08 | | | |
| 2 | N35G | S30K, T97I | 19.71 nM | 2.83E+06 | 2.49E−02 | 8.78E−09 | | | |
| 3 | N35G | L33G, T97I | 19.71 nM | 1.17E+06 | 8.27E−03 | 7.09E−09 | 1.66E+06 | 7.66E−03 | 4.60E−09 |
| 4 | N35G | K27A | 19.71 nM | 1.19E+06 | 1.07E−02 | 8.97E−09 | | | |
| 5 | N35G | S30K | 19.71 nM | 4.37E+06 | 3.45E−02 | 7.89E−09 | | | |
| 6 | N35G | L33G | 19.71 nM | 1.56E+06 | 7.96E−03 | 5.11E−09 | 1.98E+06 | 7.63E−03 | 3.86E−09 |
| 7 | N35G | T97I | 19.71 nM | 8.36E+05 | 1.41E−02 | 1.69E−08 | | | |
| 8 | T59Y | K27A, T97I | 19.71 nM | 1.24E+06 | 1.96E−02 | 1.58E−08 | | | |
| 9 | T59Y | S30K, T97I | 19.71 nM | 5.27E+06 | 6.00E−02 | 1.14E−08 | | | |
| 10 | T59Y | L33G, T97I | 19.71 nM | 2.01E+06 | 1.75E−02 | 8.70E−09 | | | |
| 11 | T59Y | K27A | 19.71 nM | 1.21E+06 | 1.71E−02 | 1.41E−08 | | | |
| 12 | T59Y | S30K | 19.71 nM | 1.52E+07 | 1.68E−01 | 1.11E−08 | | | |
| 13 | T59Y | L33G | 19.71 nM | 2.45E+06 | 2.37E−02 | 9.68E−09 | | | |
| 14 | T59Y | T97I | 19.71 nM | 7.28E+06 | 6.04E−02 | 8.30E−09 | | | |
| 15 | N35G, T59Y | K27A | 19.71 nM | 1.07E+06 | 7.55E−03 | 7.05E−09 | 1.28E+06 | 5.09E−03 | 3.98E−09 |
| 16 | N35G, T59Y | S30K | 19.71 nM | 2.13E+06 | 1.60E−02 | 7.51E−09 | 1.16E+06 | 8.08E−03 | 6.99E−09 |
| 17 | N35G, T59Y | L33G | 19.71 nM | 1.56E+06 | 6.90E−03 | 4.42E−09 | 1.23E+06 | 4.19E−03 | 3.41E−09 |
| 18 | N35G, T59Y | T97I | 19.71 nM | 1.70E+06 | 1.50E−02 | 8.83E−09 | | | |

### Example 4. In Vitro Characterization of Anti-CD24 Humanized Monoclonal Antibodies

A number of preliminary nonclinical pharmacology, pharmacokinetics and toxicity studies have been conducted with 4451HM15-IgG1-3A. 4451 HM15-IgG1-3A is a humanized antibody composed of Fab domain that recognizes the backbone amino acid of human CD24 protein and fused to a human ADCC-enhanced IgG1 Fc domain, with 3 mutations S298A/E333A/K334A. Throughout this section, 4451HM15-IgG1-3A was produced in a CHO-based mammalian expression system by Biointron.

### Sequences of 4451HM15-IgG1-3A:

| | | SEQ ID |
|---|---|---|
| Antibody chain | Amino acid sequences | NO: |
| VH | QVQLVQSGAEVKKPGASVKVSCKASGYDFTGYHMGWVRQAPGQGLEWMGEINPITSD | 111 |
| | KYFNQKFKSRVTMTRDTSISTAYMELSRLRSDDTAVYYCARRDYGTSLDYWGQGTTV | |
| | TVSS | |
| Human IgG1 | ASTKGPSVFPLAPSSKSTSGGTAALGCLVKDYFPEPVTVSWNSGALTSGVHTFPAVL | 118 |
| (S298A/E333A/ | QSSGLYSLSSVVTVPSSSLGTQTYICNVNHKPSNTKVDKKVEPKSCDKTHTCPPCPA | |
| K334A) | PELLGGPSVFLFPPKPKDTLMISRTPEVTCVVVDVSHEDPEVKFNWYVDGVEVHNAK | |
| | TKPREEQYNATYRVVSVLTVLHQDWLNGKEYKCKVSNKALPAPIAATISKAKGQPRE | |
| | PQVYTLPPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGK | |
| VL | DIQLTQSPSFLSASVGDRVTITCRASASISKYLAWYQQKPGKAPKLLIYAGSTLHSG | 106 |
| | IPSRFRGSGSGTEFTLTISSLQPEDFATYYCQQHNEYPITFGQGTKLEIK | |
| Human light chain | RTVAAPSVFIFPPSDEQLKSGTASVVCLLNNFYPREAKVQWKVDNALQSGNSQESVT | 119 |
| constant region | EQDSKDSTYSLSSTLTLSKADYEKHKVYACEVTHQGLSSPVTKSFNRGEC | |

### Soluble CD24 Binding

4451HM-IgG1-3A and two reference anti-CD24 antibodies (IMM47H and GB7011) were tested for in vitro binding with soluble recombinant CD24 protein. Since human CD24 contains only 33 core amino acids, and is heavily glycosylated, two different proteins were used to perform protein binding assay. CD24-his protein was purified from mammalian cell and as modified by posttranslational modification. While CD24-GST was purified from E. coli with no PTM. In standard ELISA, all three antibodies bound to CD24-his in a concentration-dependent manner, with EC50 of 10.12 μg/mL for 4451HM-IgG1-3A, 20.18 μg/mL for GB7011 and 27.69 μg/mL for IMM47 (FIG. 6). The results indicated that 4451HM-IgG1-3A and GB7011 bound mainly to the core protein of CD24, while IMM47 binding with CD24 is at least partially PTM-dependent.

### Cell Surface CD24 Binding

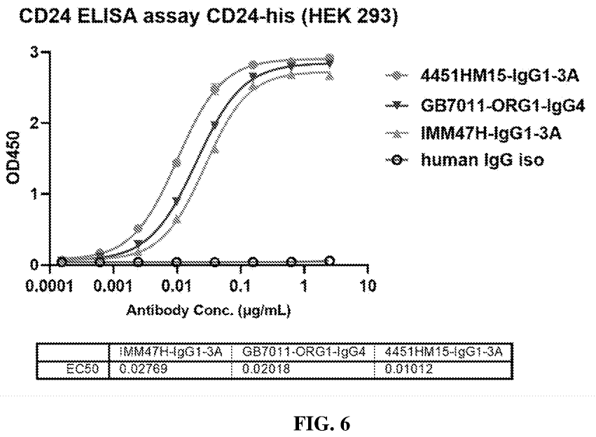

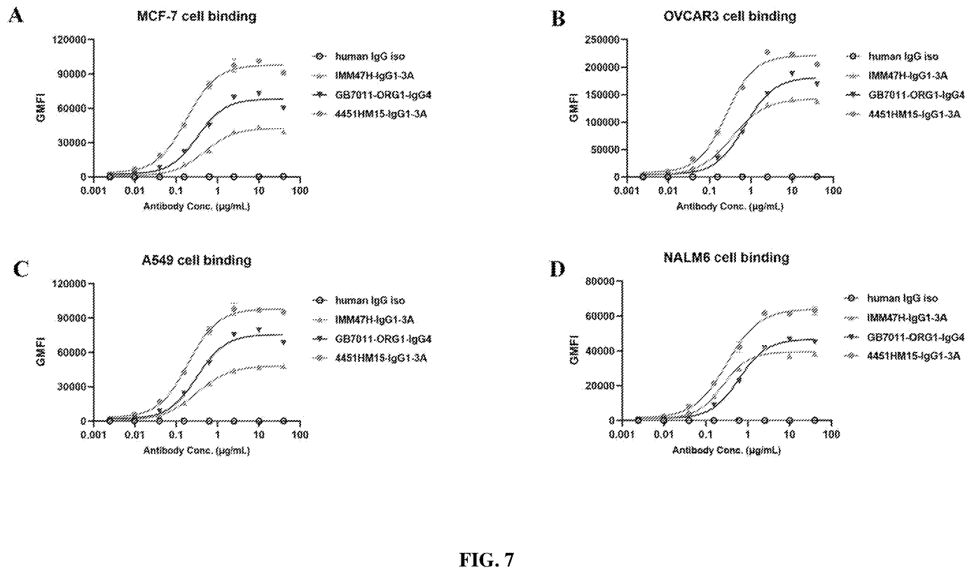

4451HM15-IgG1-3A and two reference anti-CD24 antibodies were evaluated for in vitro binding with cell-based CD24. Here we used four CD24 highly expressed tumor cell lines MCF7 (breast cancer), OVCAR3 (ovarian cancer), A549 (lung cancer), and NALM6 (B cell lymphoma). As shown in FIG. 7 (panel A-D), 4451HM15-IgG1-3A, GB7011 and IMM47H exhibited typical sigmoidal binding behavior to these cells. 4451HM15 binds to a broad cancer cells with potent activity (EC50=1.20~2.11 nM), which indicates that the binding activity of 4451HM15-IgG1-3A is more potent than GB7011 and IMM47H.

### Blocking of CD24/Siglec-10 Interaction by Anti-CD24 mAb

Anti-CD24 mAb was examined for its ability to block the binding of CD24 to its ligand Siglec-10. Here we used the human HEK293 purified CD24-his protein and biotin conjugated Siglec-10-his to conduct this assay. CD24-his protein was firstly coated in 96-well plate overnight. Then Siglec-10-his-Biotin protein and anti-CD24 mAb with a serially diluted concentration were mixed and the incubated with CD24. As shown in FIG. 8, 4451HM15-IgG4 blocked CD24/Siglec-10 interaction in a dose-dependent manner.

Phagocytosis of Tumor Cells by Human Macrophage in the Presence of Anti-CD24 mAbs

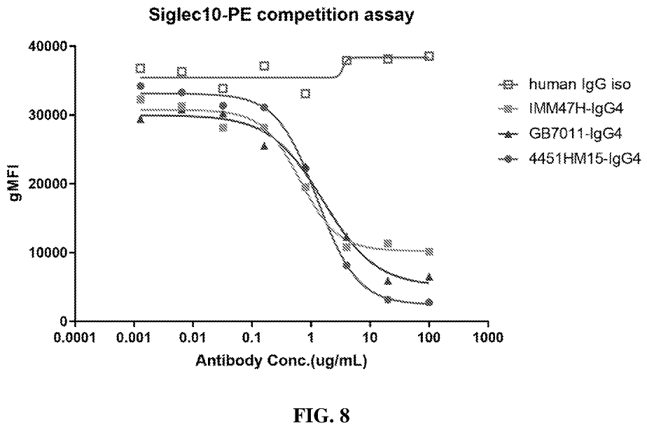

The effect of anti-CD24 mAbs on the tumor cell phagocytosis by macrophages was evaluated in an in vitro assay, in which CD24⁺ MCF7 cells were co-cultured with human differentiated macrophage in the presence or absence of anti-CD24 mAbs. Briefly, CD14⁺ monocytes were purified from human peripheral blood mononuclear cells (PBMCs) and in vitro differentiated into mature macrophages for 6 days. The monocyte derived macrophages (MDMs) were collected and re-plated in 24-well dishes overnight as effector cells. MCF7 cells as target cells were labeled with CFSE (which emits green fluorescence) and added to MDMs at a ratio of 3 tumor cells per phagocyte in the presence of different concentrations of anti-CD24 mAbs. After 1 hr incubation, non-phagocytosed target cells were washed away with PBS and the remaining phagocytes were collected and stained with macrophage marker CD14 followed by flow cytometry analysis. Phagocytosis was measured by gating on CD14⁺ cells and then assessing the percent of CFSE⁺ cells. 4451HM15-IgG1-3A significantly increased the phagocytosis of MCF7 cells and was more potent than GB7011 and IMM47H (see FIG. 9 panel A). Similar experiments were conducted in OVCAR3, NALM6 and MC38 cells (see FIG. 9 panel B-D). In conclusion, 4451HM15 enhances macrophage mediated phagocytosis against multiple tumor cells (EC50=1.97~2.45 nM).

ADCC and CDC Activity of Anti-CD24 mAbs

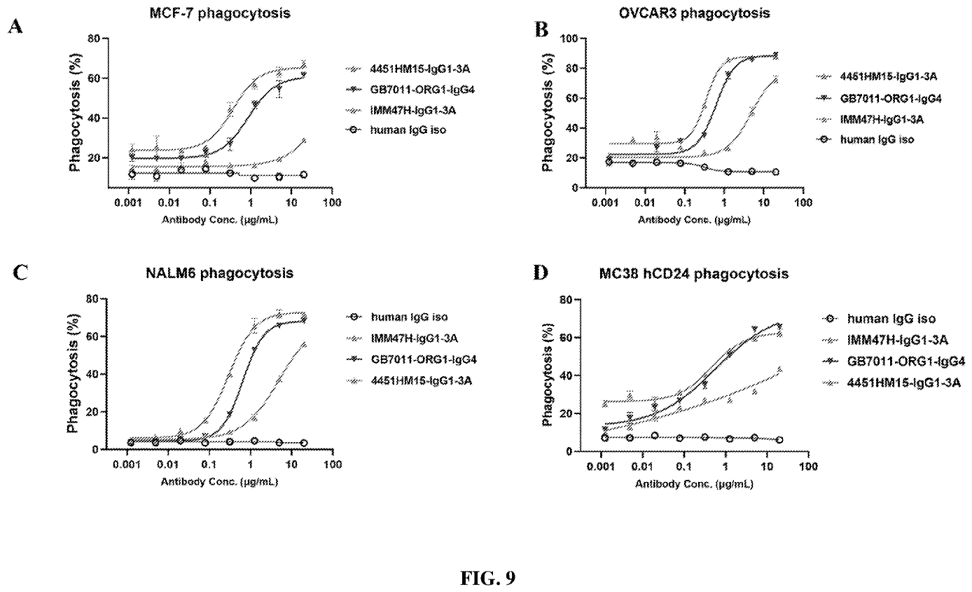

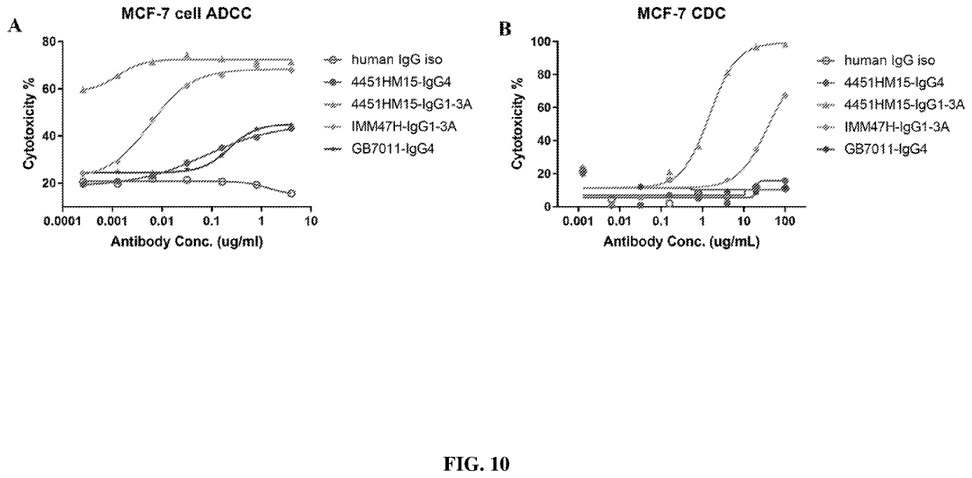

4451HM15-IgG1-3A was engineered on a human IgG1 scaffold with S298A/E333A/K334A mutations to enhance the binding to FcγR and facilitating the “eat me” signal. Also, the ability of 4451HM15-IgG1-3A to induce ADCC and CDC activity was tested against MCF7 cells. As shown in FIG. 10 (panel A-B), 4451HM15-IgG1-3A mediated ADCC and CDC in a dose-dependent manner, while its IgG4 isoform showed weak activity. 4451HM15-IgG1-3A exhibits potent ADCC (EC50<1 nM) and CDC (EC50-9.93 nM) activity compared with benchmark or 4451HM15-IgG4.

### Example 5. In Vivo Characterization of Anti-CD24 Humanized Monoclonal Antibodies

Efficacy of anti-CD24 mAb treatment in humanized Siglec-10 mice bearing hCD24 overexpressed MC38 tumor model (Batch 2 #)

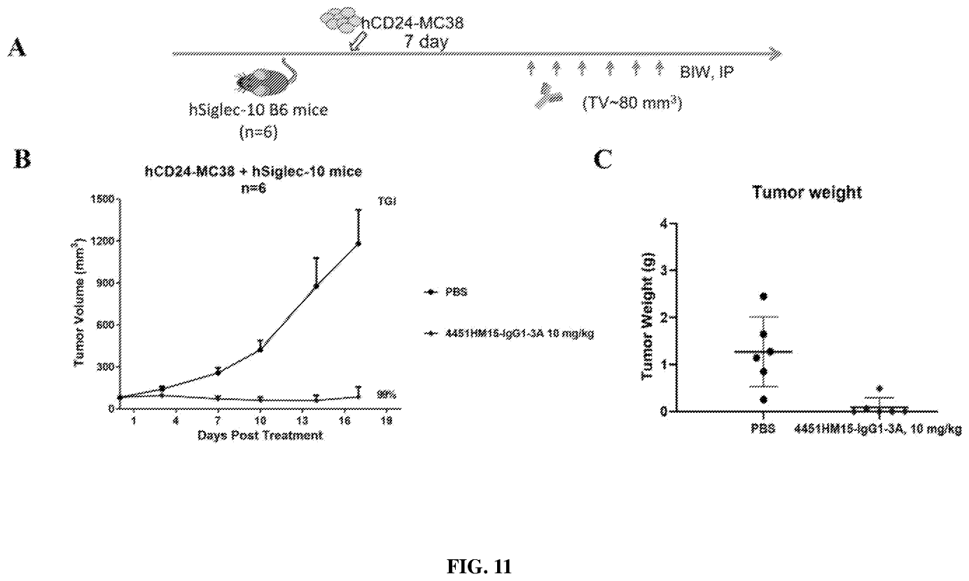

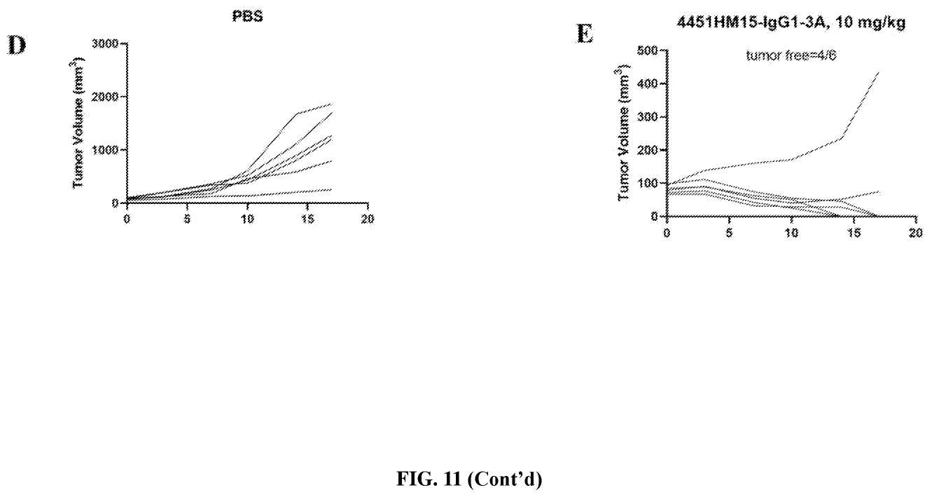

To evaluate the in vivo efficacy of final sequence 4451HM15, which optimized from 4451H by improving its binding affinity, we developed another Siglec-10 humanized syngeneic mice model. In this experiment, 4451HM15-IgG1-3A was assessed. As shown in FIG. 11 (panel A-E), 4451HM15-IgG1-3A treatment achieved 99% TGI with four mice tumor.

Efficacy of Anti-CD24 mAb Treatment in Wt C57BL/6 Mice Bearing hCD24 Overexpressed MC38 Tumor Model

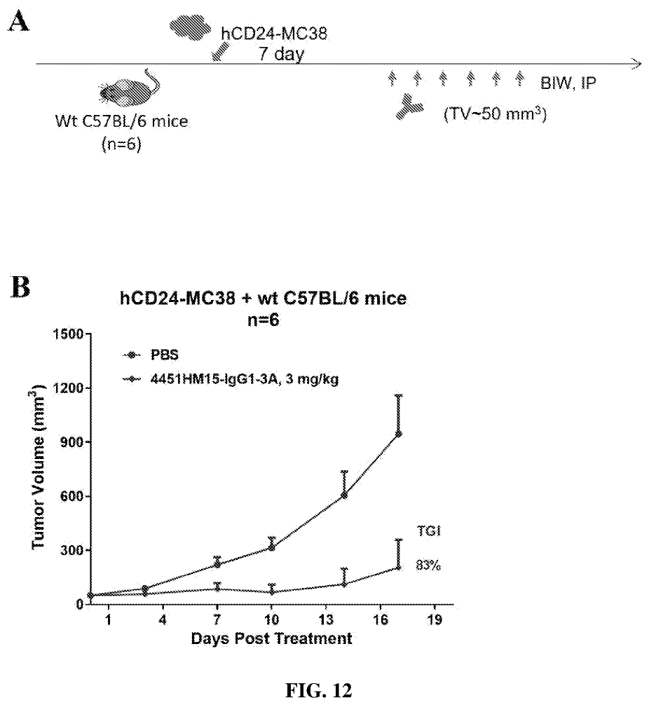

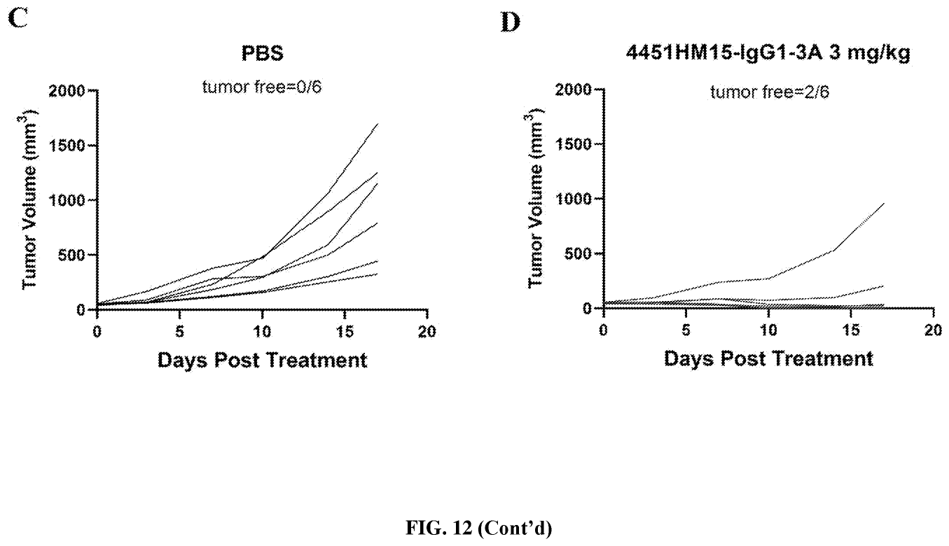

Previous research has indicated that human CD24 could interact with murine Siglec-G with weaker affinity compared with human Siglec-10. Here we also used wt C57BL/6 mice bearing human CD24 protein overexpressed MC38 tumor to test the effect of anti-CD24 mAbs. Briefly, mice were subcutaneously engrafted with hCD24-MC38 cells. When the tumor volume reached about 50 mm3, anti-CD24 antibody or PBS were intraperitoneally administrated twice a week for 3 weeks. The tumor volume was monitored twice per week during the experiment. As shown in FIG. 12 (panel A-D), 4451HM15-IgG1-3A 3 mg/kg treatment markedly reduced tumor growth compared with control group, achieving 83% TGI.

### Efficacy of Anti-CD24 mAb Treatment in Human PBMC-Reconstructed SCID Mice Bearing MCF7 Tumor Model

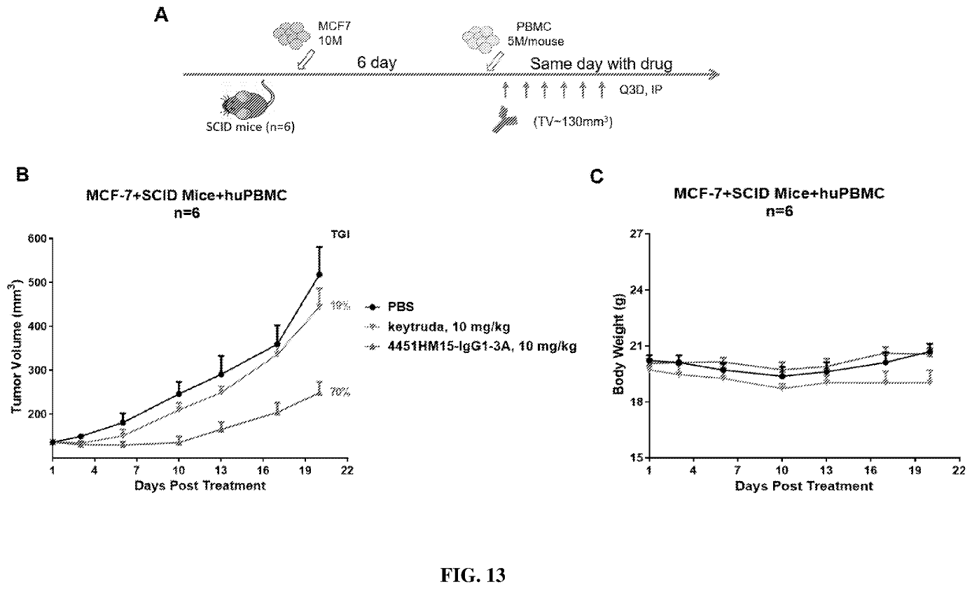

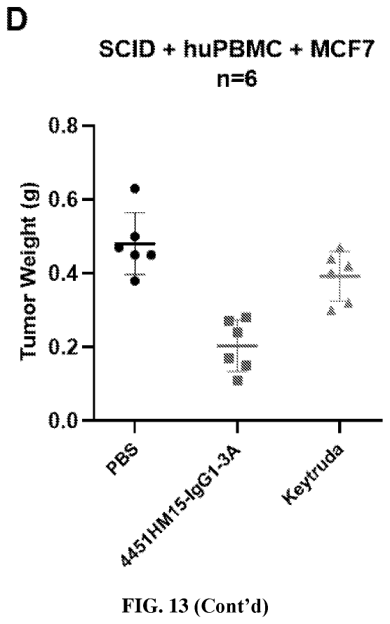

To further explore the in vivo efficacy of anti-CD24 antibodies in different tumor models, huPBMC-reconstructed SCID mice bearing MCF7 tumor was employed. Briefly, SCID mice were inoculated subcutaneously with MCF7 cells. After about a week, human PBMC was implanted via tail vein, 5 million per mouse. At the same day, anti-CD24 antibodies were intraperitoneally administrated every 3 days for 3 weeks. The tumor volume and mice weight were monitored twice per week during the experiment. As shown in FIG. 13 (panel A-D), 4451HM15-IgG1-3A treatment markedly reduced tumor growth compared with control group or Keytruda®, achieving 70% TGI.

### Efficacy of Anti-CD24 mAb Treatment in CB-17 SCID Mice Bearing SHP-77 Tumor Model

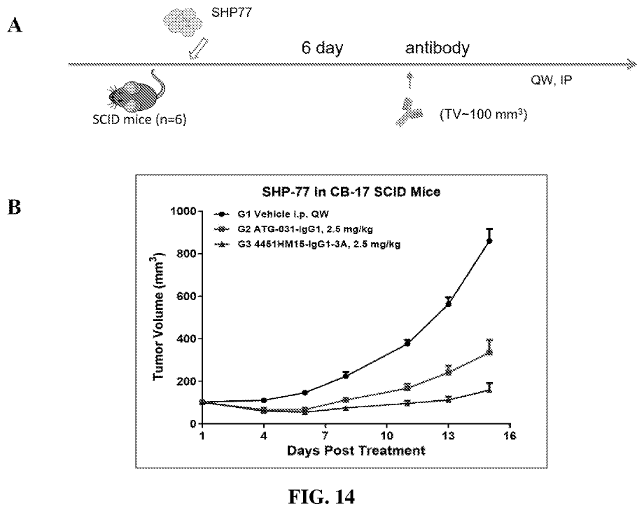

To further explore the in vivo efficacy of anti-CD24 antibodies in different tumor models, CB-17 SCID mice bearing SHP-77 tumor was employed. Briefly, mice were subcutaneously engrafted with SHP-77 cells. When the tumor volume reached about 100 mm3, anti-CD24 antibody or vehicle were intraperitoneally administrated once. The tumor volume was monitored during the experiment. As shown in FIG. 14 and Table 11, 4451HM15-IgG1-3A 3 mg/kg treatment markedly reduced tumor growth compared with control groups, achieving about 92.41% TGI.

TABLE 11
Administration regimen and TGI

| Group | N | TA | Dose (mg/kg) | TGI (%) |
|---|---|---|---|---|
| 1 | 6 | Vehicle | — | / |
| 2 | 6 | ATG031-IgG1 | 2.5 | 69.20 |
| 3 | 6 | 4451HM15-IgG1-3A | 2.5 | 92.41 |

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. An antibody or antigen-binding fragment thereof having specificity to a human CD24 protein, wherein the antibody or antigen-binding fragment thereof comprises a heavy chain variable region comprising heavy chain complementarity determining regions HCDR1, HCDR2, and HCDR3, and a light chain variable region comprising light chain complementarity determining regions LCDR1, LCDR2, and LCDR3, wherein the HCDR1, HCDR2, HCDR3, LCDR1, LCDR2, and LCDR3 comprise, respectively:
HCDR1:
(SEQ ID NO: 1)
GYHMN
or
(SEQ ID NO: 112)
GYHMG,
HCDR2:
(SEQ ID NO: 2)
EINPITSDKTFNQKFKS
or
(SEQ ID NO: 113)
EINPITSDKYFNQKFKS,
HCDR3:
(SEQ ID NO: 3)
RDYGTSLDY,
LCDR1:
(SEQ ID NO: 4)
RASKSISKYLA,
(SEQ ID NO: 114)
RASASISKYLA,
(SEQ ID NO: 115)
RASKSIKKYLA
or
(SEQ ID NO: 116)
RASKSISKYGA,
LCDR2:
(SEQ ID NO: 5)
AGSTLHS,
and
LCDR3:
(SEQ ID NO: 6)
QQHNEYPIT
or
(SEQ ID NO: 117)
QQHNEYPII.

2. The antibody or antigen-binding fragment thereof of claim 1, wherein the antibody is a chimeric antibody or a humanized antibody.

3. The antibody or antigen-binding fragment thereof of claim 1, wherein the HCDR1, HCDR2, HCDR3, LCDR1, LCDR2, and LCDR3 are HCDR1: GYHMG (SEQ ID NO: 112), HCDR2: EINPITSDKYFNQKFKS (SEQ ID NO:113), HCDR3: RDYGTSLDY (SEQ ID NO: 3), LCDR1: RASASISKYLA (SEQ ID NO: 114), LCDR2: AGSTLHS (SEQ ID NO: 5), and LCDR3: QQHNEYPIT (SEQ ID NO: 6).

4. The antibody or antigen-binding fragment thereof of claim 3, which is humanized and wherein the heavy chain variable region comprises one or more back mutations selected from the group consisting of 38K, 48I, 68A, 72V, and 74R, according to Kabat numbering, and combinations thereof.

5. The antibody or antigen-binding fragment thereof of claim 3, which is humanized and wherein the light chain variable region comprises one or more back mutations selected from the group consisting of 45R, 58I, and 63R according to Kabat numbering, and combinations thereof.

6. The antibody or antigen-binding fragment thereof of claim 3, comprising a heavy chain variable region comprising an amino acid sequence selected from the group consisting of SEQ ID NOs: 60, 94-97, 102, 110, and 111, or a peptide having at least 90% sequence identity to an amino acid sequence selected from the group consisting of SEQ ID NOs: 60, 94-97, 102, 110, and 111.

7. The antibody or antigen-binding fragment thereof of claim 3 or 6, comprising a light chain variable region comprising an amino acid sequence selected from the group consisting of SEQ ID NOs: 61, 98-101, and 103-109 or a peptide having at least 90% sequence identity to an amino acid sequence selected from the group consisting of SEQ ID NOs: 61, 98-101, and 103-109.

8. The antibody or antigen-binding fragment thereof of claim 3, comprising a heavy chain variable region comprising the amino acid sequence of SEQ ID NO: 111 or a peptide having at least 90% sequence identity to SEQ ID NO: 111, and a light chain variable region comprising the amino acid sequence of SEQ ID NO: 106 or a peptide having at least 90% sequence identity to SEQ ID NO: 106.

9. The antibody or antigen-binding fragment thereof of claim 8, comprising a heavy chain variable region comprising the amino acid sequence of SEQ ID NO: 111, and a light chain variable region comprising the amino acid sequence of SEQ ID NO: 106.

10-11. (canceled)

12. The antibody or antigen-binding fragment thereof of claim 1, further comprising a heavy chain constant region, a light chain constant region, an Fc region, or the combination thereof.

13-15. (canceled)

16. The antibody or antigen-binding fragment thereof of claim 12, wherein the Fc region is engineered to enhance the effector function.

17. The antibody or antigen-binding fragment thereof of claim 16, wherein the effector function comprises antibody-mediated cellular cytotoxicity (ADCC), complement dependent cytotoxicity (CDC) or antibody-dependent cellular phagocytosis (ADCP).

18. The antibody or antigen-binding fragment thereof of claim 17, wherein the Fc region is engineered to include S298A/E333A/K334A mutations in IgG1.

19. A bifunctional molecule, comprising a first antigen-binding portion having specificity to a human CD24 protein and a second portion having specificity to a second protein, wherein the first antigen-binding portion comprises an antibody or antigen-binding fragment thereof of claim 1.

20. A composition comprising the antibody or antigen-binding fragment thereof of claim 1, and a pharmaceutically acceptable carrier.

21. (canceled)

22. A polynucleotide encoding one or more chains of the antibody or antigen-binding fragment thereof of claim 1.

23. A method of treating a cancer in a patient in need thereof, comprising administering to the patient the antibody or antigen-binding fragment thereof of claim 1.

24. The method of claim 23, wherein the cancer is selected from the group consisting of bladder cancer, breast cancer, colorectal cancer, endometrial cancer, esophageal cancer, head and neck cancer, kidney cancer, leukemia, liver cancer, lung cancer, lymphoma, melanoma, pancreatic cancer, prostate cancer, and thyroid cancer.

25-26. (canceled)

27. A method for treating an autoimmune disease or inflammatory condition in a patient in need thereof, comprising administering to the patient the antibody or antigen-binding fragment thereof of claim 1.

28. (canceled)


SEQUENCE LISTING (119 sequences)

SEQ ID NO: 1 - protein, 5 aa, synthetic construct
GYHMN

SEQ ID NO: 2 - protein, 17 aa, synthetic construct
EINPITSDKTFNQKFKS

SEQ ID NO: 3 - protein, 9 aa, synthetic construct
RDYGTSLDY

SEQ ID NO: 4 - protein, 11 aa, synthetic construct
RASKSISKYLA

SEQ ID NO: 5 - protein, 7 aa, synthetic construct
AGSTLHS

SEQ ID NO: 6 - protein, 9 aa, synthetic construct
QQHNEYPIT

SEQ ID NO: 7 - protein, 5 aa, synthetic construct
GYYMN

SEQ ID NO: 8 - protein, 17 aa, synthetic construct
EINPITSHNTFNQKFKA

SEQ ID NO: 9 - skipped

SEQ ID NO: 10 - protein, 7 aa, synthetic construct
AGSTLQS

SEQ ID NO: 11 - protein, 9 aa, synthetic construct
QQHNEYPLT

SEQ ID NO: 12 - protein, 5 aa, synthetic construct
GYHMN

SEQ ID NO: 13 - protein, 17 aa, synthetic construct
EINPITSDKTFNQKFKA

SEQ ID NO: 14 - protein, 17 aa, synthetic construct
EVNPITGDTTYSQNFKA

SEQ ID NO: 15 - protein, 5 aa, synthetic construct
NSWMN

SEQ ID NO: 16 - protein, 17 aa, synthetic construct
QIQLKSDNYATRYVESV

SEQ ID NO: 17 - protein, 9 aa, synthetic construct
YYCTTGTDY

SEQ ID NO: 18 - protein, 16 aa, synthetic construct
KSSQSLLFSNGKTYLN

SEQ ID NO: 19 - protein, 7 aa, synthetic construct
LVSNLDS

SEQ ID NO: 20 - protein, 9 aa, synthetic construct
VQGTHFPYT

SEQ ID NO: 21 - protein, 5 aa, synthetic construct
TYWMH

SEQ ID NO: 22 - protein, 17 aa, synthetic construct
RIDPNSGETKYSEKFKN

SEQ ID NO: 23 - protein, 11 aa, synthetic construct
YYYLNNFLFDY

SEQ ID NO: 24 - protein, 12 aa, synthetic construct
RASSSVSSSYLH

SEQ ID NO: 25 - protein, 7 aa, synthetic construct
STSNLAS

SEQ ID NO: 26 - protein, 9 aa, synthetic construct
QQYSGYPLT

SEQ ID NO: 27 - protein, 5 aa, synthetic construct
KHWMN

SEQ ID NO: 28 - protein, 17 aa, synthetic construct
QIQLKSDNYAIRYVDSV

SEQ ID NO: 29 - protein, 16 aa, synthetic construct
RSSQSLLYSNGKTYLN

SEQ ID NO: 30 - protein, 7 aa, synthetic construct
VVSNLDS

SEQ ID NO: 31 - protein, 5 aa, synthetic construct
SYWLH

SEQ ID NO: 32 - protein, 17 aa, synthetic construct
RIDPNSGETKFNEKFKS

SEQ ID NO: 33 - protein, 10 aa, synthetic construct
YYYGSILFDY

SEQ ID NO: 34 - protein, 12 aa, synthetic construct
RTSSIISSGFLH

SEQ ID NO: 35 - protein, 9 aa, synthetic construct
QQYSGHPLT

SEQ ID NO: 36 - protein, 5 aa, synthetic construct
TYGMS

SEQ ID NO: 37 - protein, 17 aa, synthetic construct
WINTYSGVPAYADDFKG

SEQ ID NO: 38 - protein, 14 aa, synthetic construct
SGYPWSPHSYSTDY

SEQ ID NO: 39 - protein, 16 aa, synthetic construct
KSSQSIVHSNGNTYLE

SEQ ID NO: 40 - protein, 7 aa, synthetic construct
KVSNRFS

SEQ ID NO: 41 - protein, 9 aa, synthetic construct
FQGSHVPWT

SEQ ID NO: 42 - protein, 9 aa, synthetic construct
RDYDTSLDY

SEQ ID NO: 43 - protein, 5 aa, synthetic construct
GYYMN

SEQ ID NO: 44 - protein, 11 aa, synthetic construct
RASKSISRYLA

SEQ ID NO: 45 - protein, 7 aa, synthetic construct
AGSTLHL

SEQ ID NO: 46 - protein, 17 aa, synthetic construct
EINPITGGSTYNQKFVA

SEQ ID NO: 47 - protein, 5 aa, synthetic construct
NHWMN

SEQ ID NO: 48 - protein, 16 aa, synthetic construct
KSSQSLLYSDGKTYLN

SEQ ID NO: 49 - protein, 7 aa, synthetic construct
LVSNMDS

SEQ ID NO: 50 - protein, 17 aa, synthetic construct
EINPITGGTTYNQKFRA

SEQ ID NO: 51 - protein, 11 aa, synthetic construct
RASKSIKKYLA

SEQ ID NO: 52 - protein, 7 aa, synthetic construct
SGSTLQS

SEQ ID NO: 53 - protein, 5 aa, synthetic construct
DHWMN

SEQ ID NO: 54 - protein, 17 aa, synthetic construct
QIQLKSDNYAIRYVESV

SEQ ID NO: 55 - protein, 16 aa, synthetic construct
KSSQSLLYSNGKTYLN

SEQ ID NO: 56 - protein, 7 aa, synthetic construct
LVSKLDS

SEQ ID NO: 57 - protein, 9 aa, synthetic construct
IQGTHFPYT

SEQ ID NO: 58 - protein, 5 aa, synthetic construct
NHWMN

SEQ ID NO: 59 - protein, 17 aa, synthetic construct
QIQLKSDNYATRFAESV

SEQ ID NO: 60 - protein, 137 aa, synthetic construct
MGWSCIILFLVATATGVHSEVQLQQSGPELVKPGASVKISCKASGYDFTGYHMNWVKQSPEKSLEWIGEINPITSDKTFNQKFKSKATLTVDRSSSTAYMYLKSLTSEDSAVYYCARRDYGTSLDYWGQGTTLTVSS

SEQ ID NO: 61 - protein, 126 aa, synthetic construct
MGWSCIILFLVATATGVHSDVQITQSPSYLAASPGETITIYCRASKSISKYLAWYQEKPEKTNRLLIYAGSTLHSGIPSRFRGSGSGKDFTLTVSGLEPEDFAMYYCQQHNEYPITFGAGTKLELK

SEQ ID NO: 62 - protein, 134 aa, synthetic construct
MGWSCIILFLVATATGVHSEVKLEESGGGLVQPGGSMKLSCVASGFTFTNSWMNWVRQSPERGLDWVAQIQLKSDNYATRYVESVKGRFTISRDDSKNSVYLQMNNLRAEDTGIYYCTTGTDYWSQGTTLTVSS

SEQ ID NO: 63 - protein, 131 aa, synthetic construct
MGWSCIILFLVATATGVHSDVVVTQTPLTLSVTIGQTASISCKSSQSLLFSNGKTYLNWLFQRPGQSPKRLIYLVSNLDSGVPDRFTGSGSGTDFTLKISRVEAEDLGVYYCVQGTHFPYTFGGGTKLEIK

SEQ ID NO: 64 - protein, 139 aa, synthetic construct
MGWSCIILFLVATATGVHSQVQLQQPGADLVKPGASVKLSCKASGYTVTTYWMHWVRQRPGRGLEWIGRIDPNSGETKYSEKFKNKATLTVDRPSSTAYMQLSSLTFEDSAVYFCAIYYYLNNFLFDYWGQGTTLTVSS

SEQ ID NO: 65 - protein, 127 aa, synthetic construct
MGWSCIILFLVATATGVHSENVLTQSPAIMSASPGEKVTMTCRASSSVSSSYLHWYQKKSGASPKLWIYSTSNLASGVPARFSGSGSGTSYSLTISSVEAEDAATYYCQQYSGYPLTFGAGTKLELK

SEQ ID NO: 66 - protein, 134 aa, synthetic construct
MGWSCIILFLVATATGVHSEVNLEESGGGLVQPGGSRQLSCVASGLTFSKHWMNWVRQSPDKGLEWVAQIQLKSDNYAIRYVDSVKGRFTISRDDSENTVYLQMNNLRAEDTGIYYCTTGTDYWGQGTTLTVSS

SEQ ID NO: 67 - protein, 131 aa, synthetic construct
MGWSCIILFLVATATGVHSDVVMTQTPLTLSVTIGQPASISCRSSQSLLYSNGKTYLNWLLQRPGQSPKRLIYVVSNLDSGVPDRFIGSGSGTDFTLKISRVEAEDLGVYYCVQGTHFPYTFGGGTKLEIK

SEQ ID NO: 68 - protein, 138 aa, synthetic construct
MGWSCIILFLVATATGVHSQVQLQQPGAELVKPGASVKLSCKASGYTSTSYWLHWVKQRPGRGLEWIGRIDPNSGETKFNEKFKSQATLTVDKPSSTAYMQLNSLTSEDSAVYYCALYYYGSILFDYWGQGTTLTVSS

SEQ ID NO: 69 - protein, 127 aa, synthetic construct
MGWSCIILFLVATATGVHSENVLTQSPAIMSASPGEKVTMTCRTSSIISSGFLHWYQQKSGASPKLWIYSTSNLASGVPARFSGSGSGTSYSLTISSVEAEDAATYYCQQYSGHPLTFGAGTKLELK

SEQ ID NO: 70 - protein, 142 aa, synthetic construct
MGWSCIILFLVATATGVHSQIQLVQSGPELKKPGETVKISCKASGYTFTTYGMSWMKQAPGKGLKWMGWINTYSGVPAYADDFKGRFAFSLDTSASTAYLQINNLKNEDTATYFCARSGYPWSPHSYSTDYWGQGISVTVSS

SEQ ID NO: 71 - protein, 131 aa, synthetic construct
MGWSCIILFLVATATGVHSDVLLTQTPLSLPVSLGDQASISCKSSQSIVHSNGNTYLEWYLQKPGQSPKRLIYKVSNRFSGVPDRFSGSGSGTDFTLKISRVEAEDLGVYYCFQGSHVPWTFGGGTKLEIK

SEQ ID NO: 72 - protein, 137 aa, synthetic construct
MGWSCIILFLVATATGVHSEVQLQQSGPELVKPGASVKISCKASGYEFTGYHMNWVKQSPEKSLEWIGEINPITSDKTFNQKFKAKATLTVDRSSSTAYMHLKSLTSEDSAVYYCARRDYDTSLDYWGQGTTLTVSS

SEQ ID NO: 73 - protein, 126 aa, synthetic construct
MGWSCIILFLVATATGVHSDVQITQSPSYLAASPGETITIYCRASKSISKYLAWYQEKPEKTNRLLIYAGSTLQSGIPSRFRGSGSGKDFTLTVSGLEPEDFAMYYCQQHNEYPLTFGAGTKLELK

SEQ ID NO: 74 - protein, 137 aa, synthetic construct
MGWSCIILFLVATATGVHSEVQLQQSGPVLVNPGASVKISCKASGYSFTGYYMNWVKQSPEKSLEWIGEVNPITGDTTYSQNFKAKTTLTVDRSSSTAYMQLKSLTSEDSAVYYCTRRDYGTSLDYWGQGTTLTVSS

SEQ ID NO: 75 - protein, 126 aa, synthetic construct
MGWSCIILFLVATATGVHSDVQITQSPSYLAASPGETITIHCRASKSISRYLAWYQEKPGKINTLLIYAGSTLHLGIPSRFSGSGSGTDFTLTISSLELEDFAMYYCQQHNEYPITFGAGTKLELK

SEQ ID NO: 76 - protein, 137 aa, synthetic construct
MGWSCIILFLVATATGVHSEVQLQQSGPELVKPGASVRISCKASGYSFTGYYMNWVKQSPEKNLEWIGEINPITGGSTYNQKFVAKATLTVDRSSSTAYMQLKSLTSEDSAVYYCTRRDYDTSLDYWGQGTTLTVSS

SEQ ID NO: 77 - protein, 126 aa, synthetic construct
MGWSCIILFLVATATGVHSDVQITQSPSYLAASPGETITINCRASKSISKYLAWYQEKPGKTIKLLIYAGSTLQSGIPSRFSGSGYGTDFTLTISSLEPEDFAMYYCQQHNEYPLTFGAGTKLELK

SEQ ID NO: 78 - protein, 137 aa, synthetic construct
MGWSCIILFLVATATGVHSEVQLQQSGPELVKPGASVKISCKASGYDFTGYYMNWMKQSPEKSLEWIGEINPITSHNTFNQKFKAKATLTVDRSSSTAYMHLKSLTSEDSAVYYCARRDYGTSLDYWGQGTTLTVSS

SEQ ID NO: 79 - protein, 126 aa, synthetic construct
MGWSCIILFLVATATGVHSDVQITQSPSYLAASPGETITIYCRASKSISKYLAWYQEKPEKTIRLLIYAGSTLQSGIPSRFRGSGSGTDFTLTVSGLEPEDFAMYYCQQHNEYPLTFGAGTKLELK

SEQ ID NO: 80 - protein, 137 aa, synthetic construct
MGWSCIILFLVATATGVHSEVQLQQSGPELVKPGASVKISCKASGYEFTGYHMNWVKQSPEKSLEWIGEINPITSDKTFNQKFKAKATLTVDRSSSTAYMHLKSLTSDDSAVYYCARRDYGTSLDYWGQGTTLTVSS

SEQ ID NO: 81 - protein, 126 aa, synthetic construct
MGWSCIILFLVATATGVHSDVQITQSPSYLAASPGETITIYCRASKSISKYLAWYQEKPEKTIRLLIYAGSTLQSGIPSRFRGSGSGKDFTLTVSGLEPEDFAMYYCQQHNEYPITFGAGTKLELI

SEQ ID NO: 82 - protein, 134 aa, synthetic construct
MGWSCIILFLVATATGVHSEVRLEESGGGFVQPGGSMKLSCVASGLTFPNHWMNWVRQSPERGLAWVAQIQLKSDNYATRYVESVKGRFTISRDDSESTVYLQMNNLRAEDTGIYYCTTGTDYWGQGTTLTVSS

SEQ ID NO: 83 - protein, 131 aa, synthetic construct
MGWSCIILFLVATATGVHSDVVMTQTPLTLSLTIGQPASISCKSSQSLLYSDGKTYLNWLLQRPGQSPKRLIYLVSNMDSGVPDRFTGSGSGTDFTLKISRVEAEDLGIYYCVQGTHFPYTFGGGTKLEIK

SEQ ID NO: 84 - protein, 137 aa, synthetic construct
MGWSCIILFLVATATGVHSEVQLQQSGPELVKPGASVKISCKASGYSFTGYYMNWVKQSPEKSLEWIGEINPITGGTTYNQKFRAKATLTVDRSSSTAYMQLKSLTSEDSAVYYCTKRDYDTSLDYWGQGTTLIVSS

SEQ ID NO: 85 - protein, 126 aa, synthetic construct
MGWSCIILFLVATATGVHSDVQITQSPSYLAAFPGETITINCRASKSIKKYLAWYQEKPGKTNNLLIYSGSTLQSGIPSRFSGSGTGTDFTLTISSLEPEDFAMYYCQQHNEYPLTFGAGTKLELK

SEQ ID NO: 86 - protein, 131 aa, synthetic construct
MGWSCIILFLVATATGVHSDVVMTQTPLTLSVTIGQPASISCRSSQSLLYSNGKTYLNWLLQRPGQSPKRLIYLVSNLDSGVPDRFIGSGSGTDFTLKISRVEAEDLGVYYCVQGTHFPYTFGGGTKLEIK

SEQ ID NO: 87 - protein, 134 aa, synthetic construct
MGWSCIILFLVATATGVHSEVRLEESGGGFVQPGGSMKLSCVASGLTFPNHWMNWVRQSLERGLAWVAQIQLKSDNYATRYVESVKGRFTISRDDSKSTVYLQMNNLRPEDTGIYYCTTGTDYWGQGTTLTVSS

SEQ ID NO: 88 - protein, 137 aa, synthetic construct
MGWSCIILFLVATATGVHSEVQLQQSGPELVKPGASVKISCKASGYEFTGYHMNWVKQSPEKSLEWIGEINPITSDKTFNQKFKAKATLTVDRSSTTAYMHLKSLTSEDSAVYYCARRDYDTSLDYWGQGTTLTVSS

SEQ ID NO: 89 - protein, 126 aa, synthetic construct
MGWSCIILFLVATATGVHSDVQITQSPSYLAASPGETITIYCRASKSISKYLAWYQEKPEKTNRLLIYAGSTLQSGIPSRFRGSGSGKDFTLTVSGLEPEDFAMYYCQQHNEYPLTFGAGTKLDLK

SEQ ID NO: 90 - protein, 134 aa, synthetic construct
MGWSCIILFLVATATGVHSEVNLEESGGGLVQPGGSMEFSCVASGLTFSDHWMNWVRQSPEKGLEWVAQIQLKSDNYAIRYVESVKGRFSISRDDSKSSVYLQMNNLRAEDSGIYYCTTGTDYWGQGTTLTVSS

SEQ ID NO: 91 - protein, 131 aa, synthetic construct
MGWSCIILFLVATATGVHSDVVMTQTPLTLSVTIGQPASISCKSSQSLLYSNGKTYLNWLLQRPGQSPKRLIYLVSKLDSGVPDRFTGSGSGTDFTLKISRVEAEDLGVYYCIQGTHFPYTFGGGTKLEIK

SEQ ID NO: 92 - protein, 134 aa, synthetic construct
MGWSCIILFLVATATGVHSEVKLEESGGGLVQPGGSMKLSCVVSGLTFSNHWMNWVRQSPEKGLEWVAQIQLKSDNYATRFAESVKGRFTISRDDSKSDVYLQMNNLRTEDTGIYYCTTGTDYWGQGTTLTVSS

SEQ ID NO: 93 - protein, 131 aa, synthetic construct
MGWSCIILFLVATATGVHSDVVMTQTPLTLSVTIGQPASISCKSSQSLLYSNGKTYLNWLLQRPGQSPKRLIYLVSNLDSGVPDRFTGSGSGTDFTLKISRVEAEDLGVYYCIQGTHFPYTFGGGTKLEIK

SEQ ID NO: 94 - protein, 118 aa, synthetic construct
QVQLVQSGAEVKKPGASVKVSCKASGYDFTGYHMNWVRQAPGQGLEWMGEINPITSDKTFNQKFKSRVTMTRDTSISTAYMELSRLRSDDTAVYYCARRDYGTSLDYWGQGTTVTVSS

SEQ ID NO: 95 - protein, 118 aa, synthetic construct
QVQLVQSGAEVKKPGASVKVSCKASGYDFTGYHMNWVRQAPGQGLEWIGEINPITSDKTFNQKFKSRVTMTVDTSISTAYMELSRLRSDDTAVYYCARRDYGTSLDYWGQGTTVTVSS

SEQ ID NO: 96 - protein, 118 aa, synthetic construct
QVQLVQSGAEVKKPGASVKVSCKASGYDFTGYHMNWVRQAPGQGLEWIGEINPITSDKTFNQKFKSRATMTVDTSISTAYMELSRLRSDDTAVYYCARRDYGTSLDYWGQGTTVTVSS

SEQ ID NO: 97 - protein, 118 aa, synthetic construct
QVQLVQSGAEVKKPGASVKVSCKASGYDFTGYHMNWVKQAPGQGLEWIGEINPITSDKTFNQKFKSRATMTVDRSISTAYMELSRLRSDDTAVYYCARRDYGTSLDYWGQGTTVTVSS

SEQ ID NO: 98 - protein, 107 aa, synthetic construct
DIQLTQSPSFLSASVGDRVTITCRASKSISKYLAWYQQKPGKAPKLLIYAGSTLHSGVPSRFSGSGSGTEFTLTISSLQPEDFATYYCQQHNEYPITFGQGTKLEIK

SEQ ID NO: 99 - protein, 107 aa, synthetic construct
DIQLTQSPSFLSASVGDRVTITCRASKSISKYLAWYQQKPGKAPRLLIYAGSTLHSGVPSRFRGSGSGTEFTLTISSLQPEDFATYYCQQHNEYPITFGQGTKLEIK

SEQ ID NO: 100 - protein, 107 aa, synthetic construct
DIQLTQSPSFLSASVGDRVTITCRASKSISKYLAWYQQKPGKAPKLLIYAGSTLHSGIPSRFRGSGSGTEFTLTISSLQPEDFATYYCQQHNEYPITFGQGTKLEIK

SEQ ID NO: 101 - protein, 107 aa, synthetic construct
DIQLTQSPSFLSASVGDRVTIYCRASKSISKYLAWYQQKPGKAPRLLIYAGSTLHSGIPSRFRGSGSGTEFTLTISSLQPEDFATYYCQQHNEYPITFGQGTKLEIK

SEQ ID NO: 102 - protein, 118 aa, synthetic construct
QVQLVQSGAEVKKPGASVKVSCKASGYDFTGYHMGWVRQAPGQGLEWMGEINPITSDKTFNQKFKSRVTMTRDTSISTAYMELSRLRSDDTAVYYCARRDYGTSLDYWGQGTTVTVSS

SEQ ID NO: 103 - protein, 107 aa, synthetic construct
DIQLTQSPSFLSASVGDRVTITCRASASISKYLAWYQQKPGKAPKLLIYAGSTLHSGIPSRFRGSGSGTEFTLTISSLQPEDFATYYCQQHNEYPIIFGQGTKLEIK

SEQ ID NO: 104 - protein, 107 aa, synthetic construct
DIQLTQSPSFLSASVGDRVTITCRASKSIKKYLAWYQQKPGKAPKLLIYAGSTLHSGIPSRFRGSGSGTEFTLTISSLQPEDFATYYCQQHNEYPIIFGQGTKLEIK

SEQ ID NO: 105 - protein, 107 aa, synthetic construct
DIQLTQSPSFLSASVGDRVTITCRASKSISKYGAWYQQKPGKAPKLLIYAGSTLHSGIPSRFRGSGSGTEFTLTISSLQPEDFATYYCQQHNEYPIIFGQGTKLEIK

SEQ ID NO: 106 - protein, 107 aa, synthetic construct
DIQLTQSPSFLSASVGDRVTITCRASASISKYLAWYQQKPGKAPKLLIYAGSTLHSGIPSRFRGSGSGTEFTLTISSLQPEDFATYYCQQHNEYPITFGQGTKLEIK

SEQ ID NO: 107 - protein, 107 aa, synthetic construct
DIQLTQSPSFLSASVGDRVTITCRASKSIKKYLAWYQQKPGKAPKLLIYAGSTLHSGIPSRFRGSGSGTEFTLTISSLQPEDFATYYCQQHNEYPITFGQGTKLEIK

SEQ ID NO: 108 - protein, 107 aa, synthetic construct
DIQLTQSPSFLSASVGDRVTITCRASKSISKYGAWYQQKPGKAPKLLIYAGSTLHSGIPSRFRGSGSGTEFTLTISSLQPEDFATYYCQQHNEYPITFGQGTKLEIK

SEQ ID NO: 109 - protein, 107 aa, synthetic construct
DIQLTQSPSFLSASVGDRVTITCRASKSISKYLAWYQQKPGKAPKLLIYAGSTLHSGIPSRFRGSGSGTEFTLTISSLQPEDFATYYCQQHNEYPIIFGQGTKLEIK

SEQ ID NO: 110 - protein, 118 aa, synthetic construct
QVQLVQSGAEVKKPGASVKVSCKASGYDFTGYHMNWVRQAPGQGLEWMGEINPITSDKYFNQKFKSRVTMTRDTSISTAYMELSRLRSDDTAVYYCARRDYGTSLDYWGQGTTVTVSS

SEQ ID NO: 111 - protein, 118 aa, synthetic construct
QVQLVQSGAEVKKPGASVKVSCKASGYDFTGYHMGWVRQAPGQGLEWMGEINPITSDKYFNQKFKSRVTMTRDTSISTAYMELSRLRSDDTAVYYCARRDYGTSLDYWGQGTTVTVSS

SEQ ID NO: 112 - protein, 5 aa, synthetic construct
GYHMG

SEQ ID NO: 113 - protein, 17 aa, synthetic construct
EINPITSDKYFNQKFKS

SEQ ID NO: 114 - protein, 11 aa, synthetic construct
RASASISKYLA

SEQ ID NO: 115 - protein, 11 aa, synthetic construct
RASKSIKKYLA

SEQ ID NO: 116 - protein, 11 aa, synthetic construct
RASKSISKYGA

SEQ ID NO: 117 - protein, 9 aa, synthetic construct
QQHNEYPII

SEQ ID NO: 118 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 107 aa, synthetic construct [sequence omitted]